(19) United States
(12) Patent Application Publication
(10) Pub. No.: US 2014/0134234 A1
(43) Pub. Date: May 15, 2014

(54) GENE OR DRUG DELIVERY SYSTEM

(71) Applicants: Paul A. Grayburn, Plano, TX (US); Shuyuan Chen, Allen, TX (US)

(72) Inventors: Paul A. Grayburn, Plano, TX (US); Shuyuan Chen, Allen, TX (US)

(21) Appl. No.: 14/037,892

(22) Filed: Sep. 26, 2013

Related U.S. Application Data
(63) Continuation of application No. 11/198,210, filed on Aug. 5, 2005, now abandoned.
(60) Provisional application No. 60/599,204, filed on Aug. 5, 2004.

(51) Int. Cl. A61K 9/127

(52) U.S. Cl. CPC A61K 9/127
USPC 424/450

(57) ABSTRACT

The present invention includes compositions and methods for delivering one or more active agents in vivo by contacting a target organ or tissue with a microbubble encapsulated active agent comprising a neutrally charged lipid microbubble loaded with cationic liposomes comprising one or more active agents and selectively releasing the active agents at the target by exposing the microbubble at the target with ultrasound, wherein the active agents remain protected in the microbubble until selectively release at the target.

This application claims priority to U.S. Provisional Patent Application Ser. No. 60/599,204, filed Aug. 5, 2004.

This invention was made with U.S. Government support under Contract No. K24 HL03890 awarded by the NIH. The government has certain rights in this invention. Without limiting the scope of the invention, its background is described in connection with cationic liposome delivery of drugs.

### TECHNICAL FIELD OF INVENTION

This invention relates to compositions and methods for the delivery of active agents, and more particularly, to the controlled, localized delivery of active agents using a combination of ultrasound and microbubbles.

### BACKGROUND OF THE INVENTION

Cationic liposomes have been reported to be applicable for in vitro and in vivo delivery of macromolecules to target cells. U.S. Pat. No. 4,897,355; U.S. Pat. No. 5,334,761; and U.S. Pat. No. 6,034,137 disclose compositions and methods of use of cationic lipid aggregates, such as liposomes, unilamellar vesicles, multilamellar vesicles, and micelles, which bind negatively charged macromolecules such as DNA, RNA, protein, and small chemical compounds and upon contact with the target cell, deliver the macromolecules either inside a target cell or onto the target cell membrane. In gene transfection, the transfection efficiency with liposome delivery is reportedly high in vitro but low in vivo.

Ultrasound-mediated microbubble destruction has also been reported as an in vitro or in vivo method for delivering drugs, protein, signaling molecules or genes (including plasmid vectors or viral vectors) to specific tissues (U.S. Pat. No. 5,580,757): labeled red blood cells and polymer microspheres delivered to rat skeletal muscle (Skyba, et al. 1998; and Price, et al. 1998); oligonucleotides to dog kidney (Porter, et al. 1996); dog myocardium (Wei, et al. 1997); and cultured HeLa, NIH/3T3 and C1271 cells with chloramphenicol acetyl transferase gene (Unger, et al. 1997). In one study, recombinant adenoviral transgene containing β-galactosidase under control of a constitutive promoter was attached to the surface of albumin-coated, perfluoropropane-filled microbubbles, and delivery of the microbubbles to rat myocardium by ultrasound-mediated microbubble destruction resulted in a 10-fold increase in β-galactosidase activity compared to control animals (Shohet, et al. 2000).

In reports of ultrasound-targeted microbubble destruction, bioactive agents are either entrapped within the microbubble core using oil suspension or are attached to the microbubble shell by chemical, electrostatic or mechanical means. The microbubbles are typically about 2-4 microns in diameter and are spherical in shape. They contain a gaseous core encapsulated within a shell, wherein the gas is usually a perfluorocarbon, but air, nitrogen, or sulfur hexafluoride have also been used. The shell of the microbubble has been made of albumin, phospholipid, or polymer. According to electron microscope examination, the typical microbubble shell is about 30-50 nm thick, having the characteristics of netlike, plastic that oscillates when exposed to positive or negative pressure waves, such as ultrasound waves. Depending upon the amplitude and frequency of the applied ultrasound wave, the microbubble undergoes cavitation, to release the bioactive agent that is either encapsulated by or attached to the microbubble shell.

Even though liposome or microbubble delivery of active ingredients to target sites has been reported, these methodologies have not been as efficient in vivo as desired. In the case of delivery of bioactive DNA, there are several factors that limit transfection efficiency, hence its effectiveness. Bioactive DNA attached to the microbubble can be neutralized by circulating deoxyribonucleases (DNases). Upon release from the lipid microbubble, the DNA is free inside the target organ but may not enter the cellular membrane or the nuclear membrane. Moreover, part of the microbubble shell may remain attached to the DNA molecule and thus prevent its translation. During delivery, other types of bioactive agents are likewise susceptible to proteases, lipases, carbohydrate-cleaving enzymes, and other degradation pathways.

### SUMMARY OF THE INVENTION

It has now been found that an active ingredient such as a drug, peptide, genetic material or chemotherapeutic agent can be delivered to a target site, such as a specific organ or tissue in a mammal, with greater efficiency than has been heretofore reported. An active agent delivery system is described that includes a complex between a microbubble and a complex that includes an active agent that is pre-assembled into a liposome. The liposome complex can be disrupted at a desired time point to allow a release of the active ingredient at the target site.

The present invention also includes a method of delivering a bioactive agent to a target organ or tissue in vivo by using ultrasound-targeted microbubble destruction (UTMD), in which a neutrally charged lipid microbubble has been loaded with nanospheric cationic liposome loaded with the bioactive agent.

The present invention includes compositions and methods for delivering one or more active agents in vivo that include the steps of contacting a target organ or tissue with a microbubble encapsulated active agent having a neutrally charged lipid microbubble comprising a pre-loaded liposomes comprising one or more active agents; and selectively releasing the active agents at the target by exposing the microbubble at the target with an ultrasound, wherein the active agents remain protected in the microbubble until selectively release at the target. The active agent may include one or more molecules, e.g., a nucleic acid segment under the control of a tissue-specific promoter. Other examples include nucleic acid segment with a tissue-specific gene under the control of a tissue-specific promoter, the control of an activatable promoter, under the control of an activatable promoter that drives expression of a gene that causes apoptosis. Other examples of active agents include one or more nucleic acid segments that encodes a gene selected from the group consisting of hormone, growth factor, enzyme, apolipoprotein clotting factor, tumor suppressor, tumor antigen, viral protein, bacterial surface protein, and parasitic cell surface protein.

Generally, the microbubbles are disposed in a pharmaceutically acceptable vehicle. The active agent may be an expressible gene selected from the group consisting of, e g., mutant or wild-type: p53, p16, p21, MMAC1, p73, zac1, C-CAM, BRCAI, Rb, Harakiri, Ad E1 B, ICE-CED3 protease, IL-2, IL-3, IL-4, IL-5, IL-6, IL-7, IL-8, IL-9, IL-10, IL-11, IL-12, IL-13, IL-14, IL-15, TNF, GMCSF, β-interferon, γ-interferon, VEGF, EGF, PDGF, CFTR, EGFR, VEGFR, IL-2 receptor, estrogen receptor, Bcl-2 or Bcl-xL, ras, myc, neu, raf, erb, src, fins, jun, trk, ret, gsp, hst, abl, p53, p16, p21, MMAC1, p73, zacl, BRCAI, BRCAII, Rb, growth hormone, nerve growth factor, insulin, adrenocorticotropic hormone, parathormone, follicle-stimulating hormone, luteinizing hormone and thyroid stimulating hormone. These active agents may also include a promoter selected from the group consisting of CMV IE, LTR, SV40 IE, HSV tk, β-actin, insulin, human globin α, human globin β and human globin γ promoter and a gene under the control of the promoter.

A wide variety of ultrasound equipment and methods, frequencies, modes, energy, etc., of delivery and/or use may use used with the present invention. For example, the ultrasound may be applied in a pulsed and focused mode. The ultrasound may be applied in ultraharmonic mode, etc. Examples of microbubbles include those well known in the art, in one example, the microbubble may be a biodegradable polymer, a biocompatible amphiphilic material, a microbubbles having an outer shell comprising an outer layer of biologically compatible amphiphilic material and an inner layer of a biodegradable polymer and/or microbubbles made from amphiphilic material selected from collagen, gelatin, albumin, or globulin.

In one specific set of examples, the active agent may be a nucleic acid vector that comprises a hexokinase gene under the control of an insulin promoter, or even a nucleic acid vector that comprises a hexokinase gene I under the control of a RIP promoter. Another example of an active agent for delivery using the compositions and methods taught herein include a nucleic acid vector that comprises an hVEGF protein, an hVEGF mRNA or both an hVEGF protein and an hVEGF mRNA, or even a nucleic acid vector that comprises an hVEGF₁₆₅ protein, an hVEGF₁₆₅ mRNA or both an hVEGF₁₆₅ protein and an hVEGF₁₆₅ mRNA.

Lipids for use in making the liposomes, and their loading, are well known in the art and may include one or more of the following, e.g., 1,2-dipalmitoyl-sn-glycero-3-phosphatidylcholine and 1,2-dipalmitoyl-sn-glycero-3-phosphatidylethanolamine glycerol mixed with a vector and/plasmid. A wide variety of commercially available lipid(s), mixtures, kits and the like are well know and available.

The present invention also include a method of treating a mammal in need of such treatment by administering an effective amount of a composition with a neutrally charged lipid microbubbles loaded with cationic liposomes pre-loaded with one or more bioactive agent(s) to the mammal and releasing the bioactive agent(s) into the mammal using ultrasound. The mammal may be a patient that may be provided with the microbubble in a pharmaceutically acceptable vehicle and is exposed to ultrasound energy that is focused on the site for delivery.

Another embodiment of the present invention is a drug delivery composition for ultrasound-targeted microbubble destruction at a target site that includes a pre-assembled liposome-nucleic acid complex within and about a microbubble. The liposome-nucleic acid complex may include cationic lipids, anionic lipids or mixtures and combinations thereof. The loaded microbubbles are generally disposed in a pharmaceutically acceptable vehicle, e.g., in liquid or dry form. The microbubble may be resuspended in a pharmaceutically acceptable carrier, e.g., saline. When provided in dry for and as part of, e.g., a kit, a dry powder may be provided along with one or more disposable single or multiple use containers and delivery systems, e.g., a syringe and/or needle and may further include instructions for use. Generally, kit components will be pre-sterilized.

Pre-loaded microbubbles may be used in a method for treating a mammal in need of such treatment by providing an effective amount of a composition having a neutrally charged lipid microbubbles loaded with nanosphere cationic liposomes preloaded with a bioactive agent by disrupting the microbubbles at the target site using ultrasound-targeted microbubble destruction.

Examples of active agents include, e.g., atoms or small drugs, proteins, peptides, nucleic acids, lipids, fatty acids, carbohydrates, saccharides, polysaccharides, vitamins, minerals and combinations and mixtures thereof. Examples of nucleic acids may include ribonucleic acids, deoxyribonucleic acids, in sense or antisense orientations, linear or circular, as part of a vector having, e.g., constitutive and/or tissue-specific promoters, enhancers, silencers, homologous recombination regions, etc. Peptides may be included that are, e.g., T cell activation antigens, hormones, transmitters and the like. Proteins may be precursor proteins, antigens, antibodies, fusion proteins, structural proteins, reporters, detectable markers, enzymes (e.g., proteases, nucleases, kinases, phosphatases, metabolic enzymes) chemokines, lymphokines, interferons, interleukins, agonists, antagonists, receptors, traps and mixtures and combinations thereof. Lipids may be transmitters, components of membranes, sources of energy, agonists, antagonist, chemokines and the like. Nutritional supplements may also be delivered using the present invention, e.g., nutritionally effective amounts of DNA, protein, lipid, saccharides precursors, vitamins, minerals and the like.

Another embodiment of the present invention is a delivery composition for ultrasound-targeted microbubble destruction that includes a neutrally charged lipid microbubbles loaded with nanosphere cationic liposomes loaded with a bioactive agent.

### BRIEF DESCRIPTION OF THE DRAWINGS

For a more complete understanding of the features and advantages of the present invention, reference is now made to the detailed description of the invention along with the accompanying figures and in which:

FIG. 1 includes four panels in which the top panels are microscopic sections (100×) from a control rat (left) and a UTMD-treated rat (right). In-situ PCR hybridization was used to stain for the LacZ plasmid DNA, which is seen throughout the treated pancreas. An islet is clearly seen (arrows). Bottom panels. Sections (400×) from a control rat (left) and a rat treated with UTMD using RIP-LacZ (right). In-situ PCR hybridization was used to stain for LacZ mRNA, which is localized to the islet center.

FIG. 2 includes six panels of frozen sections of the pancreas seen under high power confocal microscopy (400×) showing an islet treated by UTMD with a DsRed plasmid under the RIP promoter. Top Panels. Images from the same islet using different filter settings to identify DsRed protein relative to beta cells. Top left panel. Presence of DsRed in islet. Top middle panel. Fluorescent antibody to insulin identifies the beta-cells in the islet center using a green filter. Top right panel. Confocal image confirms co-localization of DsRed expression to beta-cells. Bottom Panels. Images from an adjacent slice of the same islet using different filter settings to identify DsRed protein relative to alpha cells. Left bottom panel. Presence of DsRed in islet. Bottom middle panel. Fluorescent antibody to glucagon identifies the alpha-cells along the islet periphery using a green filter. Bottom right panel. Confocal image confirms that DsRed expression does not co-localize to alpha-cells.

FIG. 3 is a graph that shows whole pancreas luciferase activity in rats treated with CMV-luc (cross-hatch bars), RIP-luciferase (white bars), or RIP-luciferase plus a 20% glucose feeding for 4 days after UTMD (black bars). Glucose feeding resulted in a 4-fold up-regulation of RIP-luciferase expression, compared to RIP alone. Note the marked pancreas-specificity of luciferase expression. Only trivial activity was noted in liver and spleen, which lie along the ultrasound path. Left kidney, which is also in the path of the ultrasound beam, shows much less activity than pancreas, but does have regulatable expression of RIP-luciferase. Right kidney, which is out of the ultrasound path, shows no luciferase expression. There were 3 rats in each group. Differences in luciferase activity between organs were statistically significant by ANOVA (F=74.86, p<0.0001). Differences in plasmid (CMV vs RIP vs RIP with glucose feeding) were also statistically significant (F=42.36, p<0.0001).

FIG. 4 is a graph that shows the time course of RIP-luciferase expression. Luciferase activity declines from its peak at day 4 and is negligible by day 28. The temporal decline in luciferase activity was statistically significant by ANOVA (F=236.4, p<0.0001).

FIG. 5 includes a top panel of a Western blot showing confirming hexokinase-1 activity in isolated rat pancreas after treatment with UTMD, in normal controls, and in DsRed treated controls. Bottom left. Serum insulin levels in rats treated with hexokinase I by UTMD, DsRed control by UTMD, and sham operated controls. Group differences were significant at p=0.0033 by repeated measures ANOVA, with post-hoc Scheffe's test showing significant differences at days 5 and 10. The bottom right panel is a graph that shows serum glucose levels in rats treated with hexokinase I by UTMD, DsRed control by UTMD, and sham operated controls. Group differences were significant at p=0.0005 by repeated measures ANOVA, with post-hoc Scheffe's test showing significant differences at days 5 and 10. Data are shown as mean±one standard deviation, with n=6 (UTMD hexokinase), 3 (UTMD control) and 3 (normal control) rats per group.

FIG. 6 shows by immunoblotting the presence of hVEGF₁₆₅ protein in tissue homogenates from rat myocardium. Prominent bands consistent with hVEGF₁₆₅ are seen in all 3 rats treated with UTMD-hVEGF₁₆₅ at day 10, but only a faint band is seen in control rats (UTMD alone, hVEGF165 plasmid alone, or saline). A positive control band is also shown (+C).

FIG. 7 shows the results from RT-PCR of the presence of human VEGF165 mRNA (top panel) and rat VEGF165 mRNA (bottom panel) in tissue homogenates from rat myocardium. hVEGF165 mRNA bands are seen in the 3 rats treated with UTMD at day 5 (#1-3) and day 10 (#7-9), one rat (#14) treated with UTMD at day 30 (#13-15), but not in any control rats (#4-6, 10-12, 16-18). For display purposes, only one rat from each of the 3 control groups is shown per time period. Rat VEGF 165 mRNA targeted bands (bottom panel) are seen in all experimental rats.

FIGS. 8a-8d are histologic sections of myocardium 10 days after UTMD treatment. 8a is a low power (100×) hematoxylin-eosin staining showing a hypercellular region of myocardium. 8b is a low power (100×) image of a hypercellular region stained with anti-VEGF antibody, confirming the presence of VEGF In the hypercellular region; 8c is a high power image (400×) of hypercellular area stained with BS-lectin. Red arrows depict prominent nuclei in capillary endothelial cells, consistent with angiogenesis. There is also disorganized myocellular architecture consistent with mild inflammation; 8d is a high power (400×) image of hypercellular area stained with smooth muscle α-actin. Red arrows point to pericytes covering new blood vessels. Yellow arrows point to prominent nuclei on arteriolar smooth muscle cells. Bars indicate 100 μm.

FIG. 9 is a composite figure of the histology and a graph that shows the changes in rat myocardial capillary density after treatment. The top panels show representative sections stained with BS-lectin at 200×. Compared to control myocardium (left panel), there is an increase in capillary density in UTMD-VEGF-treated myocardium (right panel). The bottom panel is a graph that shows the mean values for capillary density (lectin+vessels<10 μm) over time following UTMD. Mean values for capillary density are remarkably stable in all controls at all 3 time points. However, in the UTMD-VEGF treated rats, capillary density is significantly increased at days 5 and 10. Error bars represent one standard deviation.

FIG. 10 is a composite figure of the histology and a graph that shows the changes in rat myocardial arteriolar density after treatment. The top panels show representative sections stained with smooth muscle α-actin at 100×. Compared to controls (left), there is an increase in arteriolar density (right). The bottom panel shows the mean values for arteriolar density (smooth muscle α-actin+vessels>30 μm) over time following UTMD. Mean values for arteriolar density are not significantly different in the controls at all three time points. However, in the UTMD-VEGF treated rats, arteriolar density is significantly increased at days 5, 10, and 30. Error bars represent one standard deviation.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used throughout the present specification the following abbreviations are used: TF, transcription factor; ORF, open reading frame; kb, kilobase (pairs); UTR, untranslated region; kD, kilodalton; PCR, polymerase chain reaction; RT, reverse transcriptase.

The term “gene” is used to refer to a functional protein, polypeptide or peptide-encoding unit. As will be understood by those in the art, this functional term includes genomic sequences, cDNA sequences, fragments and/or combinations thereof, as well as gene products, including those that may have been altered by the hand of man. Purified genes, nucleic acids, protein and the like are used to refer to these entities when identified and separated from at least one contaminating nucleic acid or protein with which it is ordinarily associated.

As used herein, the term “vector” is used in reference to nucleic acid molecules that transfer DNA segment(s) from one cell to another. The vector may be further defined as one designed to propagate a gene sequences, or as an expression vector that includes a promoter operatively linked to the gene sequence, or one designed to cause such a promoter to be introduced. The vector may exist in a state independent of the host cell chromosome, or may be integrated into the host cell chromosome

As used herein, the term “promoter” refers to a recognition site on a DNA strand to which the RNA polymerase binds. The promoter usually is a DNA fragment of about 100 to 200 basepairs (bp) in the 5′ flanking DNA upstream of the cap site or the transcriptional initiation start site. The promoter forms an initiation complex with RNA polymerase to initiate and drive transcriptional activity. The complex can be modified by activating sequences termed “enhancers” or inhibiting sequences termed “silencers.” Usually specific regulatory sequences or elements are embedded adjacent to or within the protein coding regions of DNA. The elements, located adjacent to the gene, are termed cis-acting elements. These signals are recognized by other diffusible biomolecules in trans to potentiate the transcriptional activity. These biomolecules are termed transacting factors. The presence of transacting factors and cis-acting elements contribute to the timing and developmental expression pattern of a gene. Cis acting elements are usually thought of as those that regulate transcription and are found within promoter regions and other upstream DNA flanking sequences.

As used herein, the term “leader” refers to a DNA sequence at the 5′ end of a structural gene which is transcribed along with the gene. The leader usually results in the protein having an N-terminal peptide extension sometimes called a pro-sequence. For proteins destined for either secretion to the extracellular medium or a membrane, this signal sequence, which is largely hydrophobic, directs the protein into endoplasmic reticulum from which it is discharged to the appropriate destination.

As used herein, the term “intron” refers to a section of DNA occurring in the middle of a gene which does not code for an amino acid in the gene product. The precursor RNA of the intron is excised and is therefore not transcribed into MRNA nor translated into protein.

The term “cassette” refers to the sequence of the present invention which contains the nucleic acid sequence which is to be expressed. The cassette is similar in concept to a cassette tape. Each cassette will have its own sequence. Thus by interchanging the cassette the vector will express a different sequence. Because of the restrictions sites at the 5′ and 3′ ends, the cassette can be easily inserted, removed or replaced with another cassette.

As used herein, the terms “3′ untranslated region” or “3′ UTR” refer to the sequence at the 3′ end of a structural gene which is usually transcribed with the gene. This 3′ UTR region usually contains the poly A sequence. Although the 3′ UTR is transcribed from the DNA it is excised before translation into the protein. In the present invention it is preferred to have a myogenic specific 3′ UTR. This allows for specific stability in the myogenic tissues. As used herein, the terms “Non-Coding Region” or “NCR” refer to the region which is contiguous to the 3′ UTR region of the structural gene. The NCR region contains a transcriptional termination signal. As used herein, the term “restriction site” refers to a sequence specific cleavage site of restriction endonucleases.

As used herein, the term “vector” refers to some means by which DNA fragments can be introduced into a host organism or host tissue. There are various types of vectors including plasmid, bacteriophages and cosmids.

As used herein, the term “effective amount” refers to an amount of an active agent, e.g., a gene or combination of promoter and gene delivered by UTMD into the target tissue or cells, e.g., beta cells of the pancreas, myogenic tissue or culture, angiogenic cells, etc., to produce the adequate levels of the polypeptide. One skilled in the art recognizes that this actual level will depend on the use of the MVS. The levels will be different in treatment, vaccine production, or vaccination.

“Plasmids” are designated by a lower case p preceded and/or followed by capital letters and/or numbers. The starting plasmids herein are commercially available, are publicly available on an unrestricted basis, or can be constructed from such available plasmids in accord with published procedures. In addition, other equivalent plasmids are known in the art and will be apparent to the ordinary artisan.

The term “transgene” is used herein to describe genetic material that may be artificially inserted into a mammalian genome, e.g., a mammalian cell of a living animal. The term “transgenic animal is used herein to describe a non-human animal, usually a mammal, having a non-endogenous (i.e., heterologous) nucleic acid sequence present as an extrachromosomal element in a portion of its cells or stably integrated into its germ line DNA (i.e., in the genomic sequence of most or all of its cells). Heterologous nucleic acid is introduced into the germ line of such transgenic animals by genetic manipulation of, for example, embryos or embryonic stem cells of the host animal according to methods well known in the art.

As used herein, the term “Knock-out” includes, e.g., conditional knock-outs, wherein alteration of the target gene can be activated by exposure of the animal to a substance that promotes target gene alteration, introduction of an enzyme that promotes recombination at the target gene site (e.g., Cre in the Cre-lox system), or other method for directing the target gene alteration.

As used herein, the term “knock-in” refers to an alteration in a host cell genome that results in altered expression (e.g., increased or decreased expression) of a target gene, e.g., by introduction of an additional copy of the target gene, or by operatively inserting a regulatory sequence that provides for enhanced expression of an endogenous copy of the target gene. Knock-in transgenics include heterozygous knock-in of the target gene or a homozygous knock-in of a target gene and include conditional knock-ins.

In one aspect, the present invention is a method of delivering a bioactive agent to a target organ or tissue in vivo by using an ultrasound-targeted microbubble destruction (UTMD), using microbubbles loaded with nanosphere cationic liposomes containing the bioactive agent. Exemplary microbubbles comprise but are not limited to neutrally charged lipids, polymers, metals, or acrylic shells suitable for in vivo ultrasound-targeted microbubble destruction. In one embodiment, the bioactive agent is first encapsulated within or attached to tiny cationic liposomes of nanoparticle size (10-60 nm) (hereinafter, nanosphere cationic liposomes either “loaded with” or “including” the bioactive agent refers to any bioactive agent encapsulated within or attached to the liposomes, e.g., cationic liposomes), and the liposomes are then attached to neutrally charged lipid-coated or albumin-coated microbubbles filled with a gas suitable for ultrasound microbubble destruction techniques, for example perfluoropropane. The liposomes may be attached to the outer surface of the microbubble shell, incorporated within the microbubble shell and/or encapsulated within the microbubble shell. In the present invention, one or more bioactive agents can be delivered either concomitantly or subsequently by ultrasound-targeted microbubble destruction using the neutrally charged lipid microbubbles loaded with bioactive agent-containing nanosphere cationic liposomes. In another aspect, the present invention is a method of treating a mammal in need of such treatment comprising administration of an effective amount of a composition comprising neutrally charged lipid microbubbles loaded with nanosphere cationic liposomes containing a bioactive agent via ultrasound-targeted microbubble destruction.

Examples of bioactive agents suitable for the present invention include pharmaceuticals and drugs, bioactive synthetic organic molecules, proteins, peptides, polypeptides, vitamins, steroids, polyanionic agents, genetic material, and diagnostic agents. Bioactive vitamins, steroids, proteins, peptides and polypeptides can be of natural origin or synthetic. Exemplary polyanionic agents include but are not limited to sulphated polysaccharides, negatively charged serum albumin and milk proteins, synthetic sulphated polymers, polymerized anionic surfactants, and polyphosphates. Suitable diagnostic agents include but are not limited to dyes and contrast agents for use in connection with magnetic resonance imaging, ultrasound or computed tomography of a patient.

Suitable genetic material includes nucleic acids, nucleosides, nucleotides, and polynucleotides that can be either isolated genomic, synthetic or recombinant material; either single or double stranded; and either in the sense or antisense direction, with or without modifications to bases, carbohydrate residues or phosphodiester linkages. Exemplary sources for the genetic material include but are not limited to deoxyribonucleic acids (DNA), ribonucleic acids (RNA), complementary DNA (cDNA), messenger RNA (mRNA), ribosomal RNA (rRNA), short interfering RNA (siRNA), ribozymes, and mixed duplexes and triplexes of RNA and DNA.

Genetic materials are genes carried on expression vectors including but not limited to helper viruses, plasmids, phagemids, cosmids, and yeast artificial chromosomes. The genetic material suitable for the present invention is capable of coding for at least a portion of a therapeutic, regulatory, and/or diagnostic protein. Moreover, genetic materials can preferably code for more than one type of protein. For example, a bioactive agent may comprise plasmid DNA comprising genetic material encoding therapeutic protein and a selectable or diagnostic marker to monitor the delivery of the plasmid DNA, e.g., pDsRed-human insulin promoter. Such proteins include but are not limited to histocompatibility antigens, cell adhesion molecules, growth factors, coagulation factors, hormones, insulin, cytokines, chemokines, antibodies, antibody fragments, cell receptors, intracellular enzymes, transcriptional factors, toxic peptides capable of eliminating diseased or malignant cells. Other genetic materials that could be delivered by this technique included adenovirus, adeno-associated virus, retrovirus, lentivirus, RNA, siRNA, or chemicals that selectively turn on or off specific genes, such as polyamides or peptide fragments. Modifications to wild-type proteins resulting in agonists or antagonists of the wild type variant fall in the scope of this invention. The genetic material may also comprise a tissue-specific promoter or expression control sequences such as a transcriptional promoter, an enhancer, a transcriptional terminator, an operator or other control sequences.

Examples of active agents for use with the present invention include one or more of the following therapeutics pre-loaded into a liposome and associated with microbubbles including, but are not limited to, hormone products such as, vasopressin and oxytocin and their derivatives, glucagon and thyroid agents as iodine products and anti-thyroid agents; cardiovascular products as chelating agents and mercurial diuretics and cardiac glycosides; respiratory products as xanthine derivatives (theophylline and aminophylline); anti-infectives as aminoglycosides, antifungals (e.g., amphotericin), penicillin and cephalosporin antibiotics, antiviral agents (e.g., Zidovudine, Ribavirin, Amantadine, Vidarabine and Acyclovir), antihelmintics, antimalarials, and antituberculous drugs; biologicals such as antibodies (e.g., antitoxins and antivenins), vaccine antigens (e.g., bacterial vaccines, viral vaccines, toxoids); antineoplastics (e.g., nitrosoureas, nitrogen mustards, antimetabolites (fluorouracil, hormones, progestins and estrogens agonists and/or antagonists); mitotic inhibitors (e.g., Etoposide and/or Vinca alkaloids), radiopharmaceuticals (e.g., radioactive iodine and phosphorus products); and Interferon, hydroxyurea, procarbazine, Dacarbazine, Mitotane, Asparaginase and cyclosporins, including mixtures and combinations thereof.

Other suitable therapeutics include, but are not limited to: thrombolytic agents such as urokinase; coagulants such as thrombin; antineoplastic agents, such as platinum compounds (e.g., spiroplatin, cisplatin, and carboplatin), methotrexate, adriamycin, taxol, mitomycin, ansamitocin, bleomycin, cytosine arabinoside, arabinosyl adsnine, mercaptopolylysine, vincristine, busulfan, chlorambucil, melphalan (e.g., PAM, L-PAM or phenylalanine mustard), mercaptopurine, mitotane, procarbazine hydrochloride dactinomycin (actinomycin D), daunorubicinhydrochloride, doxorubicin hydrochloride, mitomycin, plicamycin (mithramycin), aminoglutethimide, estramustine phosphate sodium, flutamide, leuprolide acetate, megestrol acetate, tamoxifen citrate, testolactone, trilostane, amsacrine (m-AMSA), asparaginase (L-asparaginase), Erwinaasparaginase, etoposide (VP-16), interferon alpha-2a, interferon alpha-2b, teniposide (VM-26), vinblastine sulfate (VLB), vincristine sulfate, bleomycin, bleomycin sulfate, methotrexate, adriamycin, and arabinosyl; blood products such as parenteral iron, hemin; biological response modifiers such as muramyldipeptide, muramyltripeptide, microbial cell wall components, lymphokines (e.g., bacterial endotoxin such as lipopolysaccharide, macrophage activation factor), sub-units of bacteria (such as Mycobacteria, Corynebacteria), the synthetic dipeptide N-acetyl-muramyl-L-alanyl-D-isog-lutamine; anti-fungalagents such as ketoconazole, nystatin, griseofulvin, flucytosine (5-fc), miconazole, amphotericin B, ricin, and beta-lactam antibiotics (e.g., penicillin, ampicillin, sulfazecin); hormones such as growth hormone, PDGF, EGF, CSF, GM-CSF, melanocyte stimulating hormone, estradiol, beclomethasone dipropionate, betamethasone, betamethasone acetate and betamethasone sodium phosphate, vetamethasonedisodiumphosphate, vetamethasone sodium phosphate, cortisone acetate, dexamethasone, dexamethasone acetate, dexamethasone sodium phosphate, flunsolide, hydrocortisone, hydrocortisone acetate, hydrocortisone cypionate, hydrocortisone sodium phosphate, hydrocortisone sodium succinate, methylprednisolone, methylprednisolone acetate, methylprednisolone sodium succinate, paramethasone acetate, prednisolone, prednisoloneacetate, prednisolone sodium phosphate, prednisolone rebutate, prednisone, triamcinolone, triamcinolone acetonide, triamcinolone diacetate, triamcinolone hexacetonide and fludrocortisone acetate; vitamins such vitamin C, E, A, K, ascyanocobalamin, neinoic acid, retinoids and derivatives such as retinolpalmitate, and alpha-tocopherol(s); peptides (e.g., T cell epitopes such as MAGE, GAGE, DAGE, etc.); proteins, such as manganese super oxide dimutase, alcohol dehydrogenase, nitric oxide synthase; enzymes such as alkaline phosphatase; anti-allergic agents such as amelexanox; anti-coagulation agents such as phenprocoumon and heparin; circulatory drugs such as propranolol; metabolic potentiators such asglutathione; antituberculars such as para-aminosalicylic acid, isoniazid, capreomycin sulfate cycloserine, ethambutol hydrochloride ethionamide, pyrazinamide, rifampin, and streptomycin sulfate; antivirals such as acyclovir, amantadine azidothymidine (AZT or Zidovudine), Ribavirin andvidarabine monohydrate (adenine arabinoside, ara-A); antianginals such asdiltiazem, nifedipine, verapamil, erythrityl tetranitrate, isosorbidedinitrate, nitroglycerin (glyceryl trinitrate) and pentaerythritoltetranitrate; anticoagulants such as phenprocoumon, heparin; antibiotics such as dapsone, chloramphenicol, neomycin, cefaclor, cefadroxil, cephalexin, cephradine erythromycin, clindamycin, lincomycin, amoxicillin, ampicillin, bacampicillin, carbenicillin, dicloxacillin, cyclacillin, picloxacillin, hetacillin, methicillin, nafcillin, oxacillin, penicillin G, penicillin V, ticarcillin rifampin and tetracycline; antiinflammatories such as difunisal, ibuprofen, indomethacin, meclofenamate, mefenamic acid, naproxen, oxyphenbutazone, phenylbutazone, piroxicam, sulindac, tolmetin, aspirin and salicylates; antiprotozoans such as chloroquine, hydroxychloroquine, metronidazole, quinine and meglumine antimonate; antirheumatics such as penicillamine; narcotics such as paregoric; opiates such as codeine, heroin, methadone, morphine and opium; cardiac glycosides such as deslanoside, digitoxin, digoxin, digitalin and digitalis; neuromuscular blockers such as atracurium besylate, gallamine triethiodide, hexafluorenium bromide, metocurine iodide, pancuronium bromide, succinylcholine chloride (suxamethonium chloride), tubocurarine chloride and vecuronium bromide; sedatives (hypnotics) such as amobarbital, amobarbital sodium, aprobarbital, butabarbital sodium, chloral hydrate, ethchlorvynol, ethinamate, flurazepam hydrochloride, glutethimide, methotrimeprazine hydrochloride, methyprylon, midazolam hydrochloride, paraldehyde, pentobarbital, pentobarbital sodium, phenobarbital sodium, secobarbital sodium, talbutal, temazepam and triazolam; local anesthetics such as bupivacaine hydrochloride, chloroprocaine hydrochloride, etidocainehydrochloride, lidocaine hydrochloride, mepivacaine hydrochloride, procainehydrochloride and tetracaine hydrochloride; general anesthetics such asdroperidol; etomidate, fentanyl citrate with droperidol, ketaminehydrochloride, methohexital sodium and thiopental sodium; and radioactive particles or ions such as strontium, iodide rhenium and yttrium, and combinations and mixtures thereof.

Prodrugs may be pre-loaded into the liposomes prior to attachment to the microbubbles. Prodrugs are well known in the art and may include inactive drug precursors that are metabolized to form active drugs. The skilled artisan will recognize suitable prodrugs (and if necessary their salt forms) as described by, e.g., in Sinkula, et al., J. Pharm. Sci. 1975 64, 181-210, the relevant portions of which are incorporated herein by reference. Prodrugs, for example, may include inactive forms of the active drugs wherein a chemical group is present on the prodrug which renders it inactive and/or confers solubility or some other property to the drug. In this form, the prodrugs are generally inactive, but once the chemical group has been cleaved from the prodrug, by heat, cavitation, pressure, and/or by enzymes in the surrounding environment or otherwise, the active drug is generated. Such prodrugs are well described in the art, and comprise a wide variety of drugs bound to chemical groups through bonds such as esters to short, medium or long chain aliphatic carbonates, hemiesters of organic phosphate, pyrophosphate, sulfate, amides, amino acids, azo bonds, carbamate, phosphamide, glucosiduronate, N-acetylglucosamine and beta-glucoside. Examples of drugs with the parent molecule and the reversible modification or linkage are as follows: convallatoxin with ketals, hydantoin with alkyl esters, chlorphenesin with glycine or alanins esters, acetaminophen with caffeine complex, acetylsalicylic acid with THAM salt, acetylsalicylic acid with acetamidophenyl ester, naloxone with sulfateester, 15-methylprostaglandin F sub 2 with methyl ester, procaine with polyethylene glycol, erythromycin with alkyl esters, clindamycin with alkylesters or phosphate esters, tetracycline with betains salts, 7-acylaminocephalosporins with ring-substituted acyloxybenzyl esters, nandrolone with phenylproprionate decanoate esters, estradiol with enolether acetal, methylprednisolone with acetate esters, testosterone with n-acetylglucosaminide glucosiduronate (trimethylsilyl) ether, cortisol or prednisolone or dexamethasone with 21-phosphate esters. Prodrugs may also be designed as reversible drug derivatives and used as modifiers to enhance drug transport to site-specific tissues. Examples of carrier molecules with reversible modifications or linkages to influence transport to a site specific tissue and for enhanced therapeutic effect include isocyanate with haloalkyl nitrosurea, testosterone with propionateester, methotrexate (3-5′-dichloromethotrexat-e) with dialkyl esters, cytosine arabinoside with 5′-acylate, nitrogen mustard (2,2′-dichloro-N-methyldiethylamine), nitrogen mustard with aminomethyltetracycline, nitrogen mustard with cholesterol or estradiol ordehydroepiandrosterone esters and nitrogen mustard with azobenzene.

The skilled art will recognize that a particular chemical group may be modified in any given drug may be selected to influence the partitioning of the drug into either the shell or the interior of the microbubbles. The bond selected to link the chemical group to the drug may be selected to have the desired rate of metabolism, e.g., hydrolysis in the case of ester bonds in the presence of serum esterases after release from the microbubbles. Additionally, the particular chemical group may be selected to influence the biodistribution of the drug employed in the microbubbles, e.g., N,N-bis(2-chloroethyl)-phosphorodiamidic acid with cyclic phosphoramide for ovarian adenocarcinoma. Additionally, the prodrugs employed within the microbubbles may be designed to contain reversible derivatives that are used as modifiers of duration of activity to provide, prolong or depot action effects.

For example, nicotinic acid may be modified with dextran and carboxymethlydextran esters, streptomycin with alginic acid salt, dihydrostreptomycin with pamoate salt, cytarabine (ara-C) with 5′-adamantoats ester, ara-adenosine (ara-A) with 5-palmirate and 5′-benzoate esters, amphotericin B with methyl esters, testosterone with 17-beta-alkyl esters, estradiol with formate ester, prostaglandin with 2-(4-imidazolyl) ethylamine salt, dopamine with amino acid amides, chloramphenicol with mono- and bis(trimethylsilyl) ethers, and cycloguanil with pamoate salt. In this form, a depot or reservoir of long-acting drug may be released in vivo from the prodrug bearing microbubbles. The particular chemical structure of the therapeutics may be selected or modified to achieve a desired solubility such that the therapeutic is loaded into a liposome prior to attaching or loading in, to, at or about a microbubble. Similarly, other therapeutics may be formulated with a hydrophobic group which is aromatic or sterol in structure to incorporate into the surface of the microbubble.

Cationic liposomes suitable for use in the present invention comprise one or more monocationic or polycationic lipids, optionally combined with one or more neutral or helper lipids. The cationic lipids suitable for the present invention can be obtained commercially or made by methods known in the art. Cationic lipids suitable for the formation of cationic liposomes are well known in the art and include but are not limited to any phospholipid-related materials, such as lecithin, phosphatidylethanolamine, lysolecithin, lysophosphatidylethanolamine, phosphatidylserine, phosphatidylinositol, sphingomyelin, cephalin, cardiolipin, phosphatidic acid, cerebrosides, dicetylphosphate, dioleoylphosphatidylcholine (DOPC), dipalmitoylphosphatidylcholine (DPPC), dioleoylphosphatidylglycerol (DOPG), dipalmitoylphosphatidylglycerol (DPPG), dipalmitoylphosphatidylethanolamine 5-carboxyspermylamide (DPPES), dioleoylphosphatidylethanolamine (DOPE), palmitoyloleoylphosphatidylcholine (POPC), palmitoyloleoylphosphatidylethanolamine (POPE) and dioleoylphosphatidyl-ethanolamine 4-(N-maleimidomethyl)cyclohexane-1-carboxylate (DOPE-mal). Additional non-phosphorous containing lipids include but are not limited to stearylamine, dodecylamine, hexadecylamine, acetyl palmitate, glycerol ricinoleate, hexadecyl stearate, isopropyl myristate, amphoteric acrylic polymers, triethanolamine-lauryl sulfate, alkyl-aryl sulfate polyethyloxylated fatty acid amides, dioctadecyldimethyl ammonium bromide and steroids such as cholesterol, ergosterol, ergosterol B1, B2 and B3, androsterone, cholic acid, desoxycholic acid, chenodesoxycholic acid, lithocholic acid, N-[1-(2,3-dioleoyloxy)propyl]-N,N,N-trimethylammonium chloride (DOTMA), 1,2-bis(oleoyloxy)-3-3-(trimethylammonia)propane (DOTAP), and 5-carboxyspermylglycine dioctadecylamide (DOGS). A preferred liposome formulation comprises the polycationic lipid 2,3-dioleyloxy-N-[2-(sperminecarboxaido)ethyl]-N,N-dimethyl-1-propanaminum trifluoroacetate (DOSPA) and the neutral lipid dioleoyl phosphatidylethanolamine (DOPE) at (3:1, w/w), and mixtures and combinations thereof.

In the method of the present invention, the cationic liposomes are loaded with the bioactive agent. In one embodiment, a cationic lipid formulation of one or more lipids dissolved in one or more organic solvents is first dried or lyophilized to remove the organic solvent(s), resulting in a lipid film. Just prior to use, the lipid film is mixed with a bioactive agent suitable for the present invention suspended in a suitable aqueous medium for forming liposomes from the dried lipid film. For example, water, an aqueous buffer solution, or a tissue culture media can be used for rehydration of the lipid film. A suitable buffer is phosphate buffered saline, i.e., 10 mM potassium phosphate having a pH of 7.4 in 0.9% NaCl solution. In another embodiment, the dried lipid film is rehydrated with a suitable aqueous medium to form liposomes before the addition of the bioactive agent. This method is preferred when the bioactive agent comprises genetic material. The incorporation of the bioactive agent into the cationic liposomes is often performed at a temperature within the range of about 0 to 30° C., e.g., room temperature, in about 5, 10-20 minutes.

In the methods of the present invention, the cationic liposomes with attached bioactive agent(s) are then loaded onto neutrally charged microbubbles. In a preferred embodiment, this is accomplished by adding to the cationic liposomes with attached bioactive agent(s) a lipid composition suitable for making the microbubble shell, mixing well, and then adding an appropriate gas for encapsulation by the microbubble shell, followed by vigorous shaking for about 5 to 60 seconds, preferably for about 20 seconds. In a preferred method, the lipid composition is kept at about 0 to 30° C. before the addition of the cationic liposomes with attached bioactive agent(s).

To form the microbubble shell, any biocompatible lipid of natural or synthetic origin known to be useful in ultrasound-targeted microbubble destruction are contemplated as part of the present invention. Exemplary lipids can be found in International Application No. WO 2000/45856 and include but are not limited to fatty acids, phosphatides, glycolipids, glycosphingolipids, sphingolipids, aliphatic alcohols, aliphatic waxes, terpenes, sesquiterpenes, and steroids. Preferable lipids are phosphocholines, phosphatidylcholines, phosphatidylethanolamines, phosphatidylserines, phosphatidylglycerols, and phosphatidylinositol. A more preferred lipid is 1,2-palmitoyl-sn-glycero-3-phosphocholine or 1,2-palmitoyl-sn-glycero-phosphatidylethanolamine. The most preferred is L-1,2-palmitoyl-sn-glycero-3-phosphocholine and L-1,2-palmitoyl-sn-glycero-phosphatidylethanolamine.

Gases suitable for the present invention are generally inert and biocompatible, including but not limited to air; carbon dioxide; nitrogen; oxygen; fluorine; noble gases such as helium, neon, argon, and xenon; sulfur-based gases; fluorinated gases; and mixtures thereof. The gas may be a perfluoropropane, e.g., octafluoropropane.

As is well known to those versed in the art, targeting ligands can also be attached to the microbubbles to confer additional tissue specificity. Such ligands could include monoclonal antibodies, peptides, polypeptides, proteins, glycoproteins, hormones or hormone analogues, monosaccharides, polysaccharides, steroids or steroid analogues, vitamins, cytokines, or nucleotides.

The delivery methods of the present invention comprising neutrally charged microbubbles loaded with nanosphere cationic liposomes containing one or more bioactive agents provide all the advantages of an ultrasound-targeted microbubble delivery system combined with all the advantages of a liposome delivery system. The ultrasound-targeted microbubble delivery system allows for delivery of a drug/gene bioactive agent to a specific organ or tissue while minimizing the exposure of other organs or tissues to the bioactive agent. During delivery, the bioactive agent(s) remain within the protective cationic liposome, which shields the bioactive agent(s) from proteases, nucleases, lipases, carbohydrate-cleaving enzymes, free radicals, or other chemical alterations. This method increases the delivery of the bioactive agent and its bioavailability to the target tissue. For example, in the delivery of neutrally charged microbubbles loaded with nanosphere cationic liposomes containing plasmid DNA, the level of gene expression at the target site is increased over the level of expression possible with either a microbubble delivery or a liposome delivery of the same plasmid DNA.

In one aspect, the present invention is a method of treating a mammal in need of such treatment comprising administration of an effective amount of a composition comprising neutrally charged lipid microbubbles loaded with nanosphere cationic liposomes containing a bioactive agent via ultrasound-targeted microbubble destruction. Administration of the composition comprising neutrally charged lipid microbubbles loaded with nanosphere cationic liposomes containing a bioactive agent and the ultrasound-targeted microbubble destruction of these microbubbles to release the bioactive agent can be accomplished by any means known in the art. Repeat administration of the microbubbles is possible, particularly to prolong the duration of the therapeutic effect. For example, repeated transfection of cardiomyocytes by ultrasound targeted microbubble destruction has been shown to extend the peak duration of luciferase activity in the heart from 4 days to 12 days (Bekeredjian et al, 2003). This potentially allows for the duration of gene or drug delivery to be tailored to the specific biological or medical need.

The compositions and methods of use of the present invention are further illustrated in detail in the examples provided below, but these examples are not to be construed to limit the scope of the invention in any way. While these examples describe the invention, it is understood that modifications to the compositions and methods are well within the skill of one in the art, and such modifications are considered within the scope of the invention.

### Example 1

Preparation of Cationic Liposome Solution. Loaded with Bioactive Ingredient. To prepare cationic liposome solution loaded with the plasmid DNA pCMV-luc, 50-100 microliters containing 2 milligrams of plasmid DNA was added just prior to use to 50 microliters of cationic liposome solution (Lipofectamine 2000; Invitrogen, Carlsbad, Calif.) and incubated for 10-20 minutes at room temperature. The resulting liposomes encapsulated the plasmid DNA and were roughly 250 nanometers in diameter. The liposomes can be stored at −20 degrees C. for later use.

### Example 2

Preparation of Microbubble Formula Containing Plasmid DNA. A microbubble formula (hereinafter referred to as “Formula 2”) that incorporated plasmid DNA pCMV-luc within the microbubble shell was prepared according to a modification of a previously described method of Unger et al. (Unger, et al. 1997. “Ultrasound enhances gene expression of liposomal transfection,” Invest Radiol 32:723-727; U.S. Pat. No. 6,521,211). Briefly, in a sealable tube, 250 microliters of 2% 1,2-dipalmitoyl-sn-glycero-3-phosphocholine (C16) dissolved in PBS and prewarmed to 42 degrees C. was mixed with 1 milligram plasmid DNA pCMV-luc and incubated for 30 minutes at 40 degrees C. PBS was added as needed to achieve a total final volume of 500 microliters. The tube was then filled with octafluoropropane gas and shaken vigorously for 20 seconds in a dental amalgamator (VIALMIX®; Briston-Myers Squibb Medical Imaging, Inc., North Billerica, Mass.). The liquid subnatant comprising unattached DNA pCMV-luc was removed and discarded, leaving a milky-white supernatant layer of the lipid-coated microbubble suspension. The resulting microbubble suspension was then diluted 1:1 with PBS prior to infusion.

### Example 3

Preparation of Neutrally Charged Lipid Microbubbles Loaded with Nanosphere Cationic Liposomes Containing Plasmid DNA. A neutrally charged lipid microbubble loaded with a cationic liposome/DNA complex (hereinafter referred to as “Formula 1”) was prepared as follows. To a tube containing 50 microliters of the loaded cationic liposome/DNA complex prepared as given in Example 1 was added 250 microliters of 2% 1,2-dipalmitoyl-sn-glycero-3-phosphocholine (C16) (prewarmed to 42 degrees C.) and 5 microliters of 10% albumin solution and 50 microliters of glycerol. The mixture was mixed well but gently using a pipette. PBS was added as needed to achieve a total final volume of 500 microliters. The tube containing the mixture was then filled with octafluoropropane gas and shaken vigorously in a dental amalgamator (VIALMIX®) for 15-35 seconds at 0-4 degrees C. During this process, the plasmid DNA was first encapsulated within the cationic liposomes, and then the loaded cationic liposomes were attached to the microbubble shell. The resulting microbubble suspension was then diluted 1:1 with PBS prior to infusion.

### Example 4

Comparison of Neutrally Charged Lipid Microbubbles Loaded with Nanosphere Cationic Liposomes Containing Plasmid DNA (Formula 2) and Microbubble Formula Containing Plasmid DNA (Formula 1). The physical characteristics of the microbubbles loaded with nanosphere cationic liposomes containing plasmid DNA prepared according to the method of Example 3 (“Formula 1”) were compared to the microbubble formula containing plasmid DNA prepared according to the method of Example 2 (“Formula 2”). The bubble size and concentration of microbubbles were measured by Coulter counter. To measure the DNA loading amount of the microbubbles, each formula was washed three times with PBS to remove unattached DNA pCMV-luc. The DNA was extracted with from the microbubbles with chloroform:phenol:isopropanol (25:24:1); the DNA concentration was measured by optical density at a wavelength of 260 nm; and the integrity of the DNA was confirmed by gel electrophoresis. For the Formula 2 microbubbles, confocal microscopy using fluorescent labeled plasmid was used to confirm that the plasmid DNA was incorporated into the phospholipid shell of the microbubbles. For Formula 1, confocal microscopy using fluorescent labeled plasmid was used to confirm that the plasmid DNA was incorporated into liposomes attached to the phospholipid shell of the microbubbles. According to the results summarized in Table I, there was considerable improvement in the amount of DNA loaded into the microbubbles loaded with nanosphere cationic liposomes containing plasmid DNA (Formula 1) compared to the amount of DNA loaded into the microbubbles containing plasmid DNA (Formula 2).

TABLE 1
Physical Characteristics of Microbubble Formulations

| | | | DNA (pg/each |
|---|---|---|---|
| Formula | Bubble size (μm) | Concentration (per ml) | microbubble) |
| 1 | 2.16 ± 0.34 | 5.25 ± 0.125 × 10⁹ | 76 |
| 2 | 1.94 ± 0.26 | 1.29 ± 0.178 × 10⁹ | 1.26 |

### Example 5

In Vivo Studies in Rats: Delivery of Neutrally Charged Lipid Microbubbles Loaded with Nanosphere Cationic Liposomes Loaded with Plasmid DNA pCMV-luc (Formula 1) or Microbubble Formula Containing Plasmid DNA pCMV-luc (Formula 2). The delivery of plasmid DNA in vivo by ultrasound-mediated microbubble destruction was examined using Sprague-Dawley male rats weighing 200-300 g. In one experimental group, the gene delivery vehicle was neutrally charged lipid microbubbles loaded with nanosphere cationic liposomes loaded with plasmid DNA pCMV-luc prepared according to procedures in Example 3. In a second experimental group, the gene delivery vehicle was the microbubble formula loaded with plasmid DNA pCMV-luc prepared according to procedures in Example 2. The following procedure was performed for each experimental group with three rats in each group.

Rats weighing between 200-300 grams were anesthetized with 2-3 ml of 4× Avertin (2 gram of 2,2,2-tribromethanol and 1.24 ml 2-methyl-2-butanol in 38.76 ml H₂O) i.p. Once anesthetized, all hair on the chest and neck of the rats was removed. A 5 mm incision was made above the jugular vein medio-lateral to the neck, and a catheter was inserted into the jugular vein by cutdown. EKG probes were attached to three paws for monitoring, 1-2 centimeters of acoustic coupling gel was applied to the chest, and an S3 transducer was clamped to the chest on top of the acoustic coupling gel. Echocardiography was performed using an S12 transducer (Sonos 5500, Philips Ultrasound, Andover, Mass.) to locate the heart and record left ventricle function in a mid short axis view, with the myocardium and cavity clearly distinguishable. One milliliter of microbubble suspension was infused at a constant rate of 3 mL/h into the rat's jugular vein using an infusion pump connected to the catheter over a 15-20 minute period. During microbubble infusion, the S3 transducer clamped to the rat's chest was operated in ultraharmonic mode (settings: transmit ⅓ MHz and receive 3.6 MHz; mechanical index 1.6; depth at 3 cm; triggered imaging at every fourth heartbeat; delay of 80 ms after the peak of the R wave; all segmental gains to 0; receive gain at 50; compression at 75; and linear post-processing curve) to target microbubble destruction to the heart. The rat left ventricle was monitored at every fourth heartbeat before and after high mechanical index ultrasound.

An exemplary reading showing a rat left ventricle in triggered harmonic mode during microbubble infusion in a mid-short axis view, with the left view showing the left ventricle before high mechanical index ultrasound and the right view showing the left ventricle after high mechanical index ultrasound (data not shown). Destruction of the microbubbles was indicated by the lowering of opacification of the myocardium.

After the study, the catheter was removed, the incision sutured and the animal allowed to awaken. After 4 days, the rats were sacrificed; the atria, liver, lung, and hindlimb skeletal muscle were harvested as positive and negative controls. The left ventricle was isolated by careful dissection, then divided into anterior and posterior sections. All tissues were snap frozen with liquid nitrogen and stored at −70 degrees C. until assayed for luciferase activity.

### Example 6

In Vivo Studies in Rats: A Comparison of Luciferase Activity of Neutrally Charged Lipid Microbubbles Loaded with Nanosphere Cationic Liposomes Containing Plasmid DNA pCMV-luc (Formula 1) and Microbubble Formula Containing Plasmid DNA pCMV-luc (Formula 2). Using a luciferase assay previously described (Chen, 2003), the expression of the transgene was determined for each tissue isolated as given in Example 5: the anterior left ventricle, posterior left ventricle, atria, liver, lung, and hindlimb skeletal muscle. Each tissue was pulverized with a mortar and pestle and then disrupted with a Polytron in luciferase lysis buffer (0.1% NP-40, 0.5% deoxycholate and proteinase inhibitors, Promega Corp., Madison, Wis.). The resulting homogenate was centrifuged at 10,000 g for 10 minutes, and 100 microliters of luciferase reaction buffer (Promega) was added to 20 microliters of the clear supernatant. Light emission was measured by a luminometer (TD 20/20, Turner Designs, Inc., Sunnyvale, Calif.) in relative light units (RLU) per minute. Total protein content was determined by a modification of the Lowry method using a commercial kit (Pierce Endogen; Rockford, Ill.)(Brown, 1989). As shown in Table II, the results indicate increased delivery of the plasmid DNA to the atria, anterior left ventricle, posterior left ventricle, and lungs for microbubbles loaded with cationic liposomes containing the plasmid DNA. Essentially no delivery was observed in the liver and muscle, indicating that the ultrasound-targeted microbubble destruction technique achieved organ specificity with plasmid DNA.

### Example 7

Preparation and Characterization of Various Neutrally Charged Lipid Microbubbles Loaded with Nanosphere Cationic Liposomes Containing Plasmid DNA. Using the procedure given in Example 3, neutrally charged lipid microbubbles loaded with a cationic liposome/DNA complex were prepared using either 2% 1,2-diphenoyl-sn-glycero-phosphocholine (Formula 1-C12), 2% 1,2-dipalmitoyl-sn-glycero-phosphocholine (Formula 1—C16), or 2% 1,2-didecanoyl-sn-glycero-phosphocholine (Formula 1—C20).

TABLE 2
Luciferase Activity for Microbubbles Loaded with Cationic Liposomes Containing Plasmid
DNA pCMV-luc and Microbubble Formula Containing Plasmid DNA pCMV-luc
Luciferase activity for each isolated tissue
RLU/mg. Protein/min

| | Atria | anterior LV | posterior LV | Lung | Liver | Muscle |
|---|---|---|---|---|---|---|
| Formula 1: | | | | | | |
| Rat 1 | 724 | 22496 | 12478 | 165.2 | 7.8 | 0.4 |
| Rat 2 | 501 | 35423 | 16883 | 148.8 | 13.7 | 0.4 |
| Rat 3 | 903 | 29372 | 7137 | 112.2 | 16.8 | 0.2 |
| mean | 709.2 ± 164.2 | 29097 ± 5281 | 12166 ± 3985 | 142.7 ± 22.2 | 12.8 ± 3.7 | 0.3 ± 0.1 |
| Formula 2: | | | | | | |
| Rat 4 | 149 | 2632 | 1410 | 1.1 | 2.1 | 0.1 |
| Rat 5 | 182 | 2219 | 1890 | 2 | 2.9 | 0.2 |
| Rat 6 | 116 | 2662 | 1171 | 1.7 | 2.8 | 0.1 |
| mean | 149 ± 33 | 2504 ± 247 | 1490 ± 366 | 1.6 ± 0.4 | 2.6 ± 0.1 | 0.1 ± 0.1 |

Formula 1 = microbubbles loaded with cationic liposomes containing pCMV-luc as prepared in Example 3; Formula 2 = microbubble formula containing pCMV-luc as prepared in Example 2; LV = left ventricle.

The physical characteristics of the respective microbubbles were measured as given in Example 4 and are summarized in Table III. The bubble size and concentration per milliliter of all three formulae were similar. The amount of DNA per microbubble increased as the number of carbons increased: C20>C16>C12.

Each microbubble formula was administered to rats according to the procedure given in Example 5, with 2 rats in each experimental group. A luciferase assay was performed on harvested tissue according to the procedure in Example 6, and the results are presented in Table IV. Treatment with Formula 1-C16 resulted in greater delivery of the plasmid DNA to the target tissues.

TABLE 3
Physical Characteristics of Microbubble Formulations

| | | | DNA (pg/each |
|---|---|---|---|
| Formula | Bubble size (μm) | Concentration (per ml) | microbubble) |
| 1-C12 | 1.75 ± 0.24 | 2.78 ± 0.48 × 10⁹ | 0.26 |
| 1-C16 | 2.16 ± 0.34 | 5.25 ± 0.125 × 10⁹ | 76 |
| 1-C20 | 1.92 ± 0.42 | 1.92 ± 0.24 × 10⁹ | 128 |

TABLE 4
Luciferase Activity for Microbubble Formulae Loaded with Cationic Liposomes
Containing Plasmid DNA pCMV-luc
Luciferase activity for each isolated tissue
RLU/mg. Protein/min

| Formula: | Atria | anterior LV | posterior LV | Lung | Liver | Muscle |
|---|---|---|---|---|---|---|
| 1 - C12 | 48.9 ± 26 | 617 ± 421 | 195 ± 86 | 0.6 ± 0.4 | 0.6 ± 0.1 | 0.1 ± 0.1 |
| 1 - C16 | 709 ± 112 | 29097 ± 4823 | 12165 ± 7831 | 142 ± 65 | 12 ± 8 | 0.3 ± 0.1 |
| 1 - C20 | 440 ± 120 | 2760 ± 1262 | 1310 ± 821 | 29 ± 11 | 10.5 ± 6 | 0.1 ± 0.1 |

Formula 1 - C12 = neutrally charged lipid microbubbles loaded with a cationic liposome/DNA complex made with 2% 1,2-diphenoyl-sn-glycero-phosphocholine (C12); Formula 1 - C16 = neutrally charged lipid microbubbles loaded with a cationic liposome/DNA complex made with 2% 1,2-dipalmitoyl-sn-glycero-phosphocholine (C16); Formula 1 - C20 = neutrally charged lipid microbubbles loaded with a cationic liposome/DNA complex made with 2% 1,2-didecanoyl-sn-glycero-phosphocholine (C20); LV = left ventricle

### Example 8

Preparation and Characterization of OPTISON™ Loaded with Cationic Liposomes Loaded with Plasmid DNA pCMV-luc. A OPTISON™ (Amersham Health, Princeton, N.J.) microbubble loaded with cationic liposome/plasmid DNA complex (hereinafter referred to as “Optison Formula”) was prepared as follows. To a 1.5 ml centrifugation tube was added 1.5 ml of OPTISON™ suspension (OPTISON™ contains per ml 5.0 to 8.0×10⁸ human albumin microspheres; 10 mg albumin human, USP; 0.22±0.11 mg/mL octafluoropropane; 0.2 mg N-acetyltryptophan; and 0.12-mg caprylic acid in 0.9% aqueous sodium chloride). The OPTISON™ suspension was centrifuged at 1000 rpm for 1 minute, and the subnatant was removed and discarded. Just prior to use, 2 milligrams of plasmid DNA pCMV-luc was added to 100 microliters of cationic liposome solution (Lipofectamine 2000; Invitrogen, Carlsbad, Calif.) and incubated for 15 minutes at room temperature. The resulting cationic liposome/plasmid DNA complex was added to the OPTISON™ supernatant, and the mixture was mixed well but gently using a pipette. The tube containing the mixture was then filled with octafluoropropane gas and shaken vigorously with a dental amalgamator for 20 seconds. The resulting Optison Formula had the DNA-containing liposomes attached to an albumin shell.

The Optison Formula was administered to rats according to the procedure given in Example 5, with 3 rats in the experimental group. A luciferase assay was performed on harvested tissue according to the procedure in Example 6, and the results are presented in Table V. Treatment with Formula 1-C16 resulted in greater delivery of the plasmid DNA to the target tissues. Increased protein expression was obtained in the target tissues, although expression levels did not reach that observed with the microbubbles prepared with phospholipids.

### Example 9

Preparation and Activity of Neutrally Charged Lipid Microbubbles Loaded with Nanosphere Cationic Liposomes Containing Plasmid DNA pDsRed-RIP. Neutrally charged lipid microbubbles loaded with cationic liposomes containing pDsRed-RIP were prepared according to the procedure given in Example 3, with the substitution of the plasmid DNA.

TABLE 5
Luciferase Activity for OPTISON ™ Microbubble Formula Loaded with Cationic
Liposomes Containing Plasmid DNA pCMV-luc
Luciferase activity for each isolated tissue
RLU/mg. Protein/min

| | Atria | anterior LV | posterior LV | Lung | Liver | Muscle |
|---|---|---|---|---|---|---|
| Rat 1 | 28.6 | 2585.7 | 802.5 | 28.9 | 39.6 | 5.1 |
| Rat 2 | 24.4 | 3872.2 | 1503.6 | 9.4 | 6.2 | 2.9 |
| Rat 3 | 58.5 | 3852.6 | 2910.7 | 28.1 | 9.0 | 6.2 |
| mean | 37.2 ± 18.6 | 3436.8 ± 737.2 | 1738.8 ± 1073 | 22.1 ± 11.0 | 18.3 ± 18.5 | 4.7 ± 1.7 |

LV = left ventricle

Using a modified version of the ultrasound-targeted microbubble destruction technique outlined in Example 5, the new formula was delivered to rat pancreas. The results showed transfection of 70% of islets in the pancreas and that the transfection was specific to beta-cells (insulin-producing cells).

### Example 9

Efficient Gene Delivery to Pancreatic Islets with Ultrasonic Microbubble Destruction Technology. This example describes a novel method of gene delivery to pancreatic islets of adult, living animals by ultrasound-targeted microbubble destruction (UTMD) technology. The technique involves incorporation of plasmids into the phospholipid shell prior to loading gas-filled microbubbles. The complex was then infused into rats and destroyed within the pancreatic microcirculation using ultrasound. Specific delivery of genes to islet beta-cells by UTMD was achieved by use of a plasmid containing a rat insulin promoter (RIP), and reporter gene expression was regulated appropriately by glucose in animals that received a RIP-luciferase plasmid. To demonstrate biological efficacy, UTMD was used to deliver a RIP-hexokinase I plasmid. This resulted in a clear increase in hexokinase I protein expression in islets, increased insulin levels in blood, and a decrease in circulating glucose levels. In sum, the UTMD vesicle and construct described herein allowed delivery of genes specifically to pancreatic islets with sufficient efficiency to modulate beta-cell function in living animals.

Both major forms of diabetes involve beta-cell destruction and dysfunction. Type 1 diabetes, which afflicts approximately 1 million patients in the United States,¹ is a condition of complete insulin deficiency brought about by autoimmune destruction of the insulin producing islet beta-cells. Type 2 diabetes afflicts 16 million Americans,¹ and the hyperglycemia associated with this disease develops when insulin secretory capacity can no longer compensate for peripheral insulin resistance. Potential new treatments for both forms of diabetes could be developed if it were possible to deliver genes or other molecular cargo to pancreatic islets to enhance insulin secretion or beta-cell survival.² While viral vectors have been used for efficient gene transfer to pancreatic islets ex vivo,^{3,4} in vivo targeting to beta-cells has not been successful because of the difficulty in traversing the endothelial barrier. Moreover, most viral gene transfer vectors⁵ are limited by hepatic toxicity, immunogenic properties, inflammation, and low tissue specificity, as well as the difficulty and expense of producing large amounts of pure virus. The use of naked DNA or liposome carriers has the disadvantage of low transfection efficiency and the requirement for invasive delivery by direct injection.

A novel technique was developed that employs ultrasound-targeted microbubble destruction (UTMD) to deliver genes or drugs to specific tissues.⁶⁻¹¹ Briefly, genes are incorporated into cationic liposomes and then attached or loaded to the phospholipid or albumin shell of gas-filled microbubbles to form a delivery vehicle-microbubble complex. The delivery vehicle-microbubble complex was then injected intravenously and destroyed within the microvasculature of the target organ by ultrasound. The compositions and methods taught here were also used to enhance tissue specificity (see other examples), such as decorating the microbubbles with cell-specific ligands,¹² the use of cell-specific¹³ or pathology-specific¹⁴ promoters in transgene construction, and physical placement of the vector in the target tissue by catheter-based methods^{15,16} or direct injection.¹⁷⁻¹⁹

UTMD has been used to target reporter genes and VEGF-mediated angiogenesis to rat myocardium (see example below).⁴⁻⁷ The present invention demonstrates safe and successful targeting of reporter genes to pancreatic islets, using the rat insulin promoter to achieve a high level of islet and beta-cell specificity, as well as regulation of the delivered transgene within the islets by glucose feeding. Moreover, beta-cell specific delivery of the hexokinase-1 gene by UTMD results in increased insulin secretion. These data shows that UTMD delivers transgenes to islet beta-cells of adult, living animals at a level sufficient to alter beta-cell function, thereby providing a potential means for targeting therapeutic agents to the islets in the setting of diabetes.

Briefly, plasmid DNA with the reporter genes LacZ, DsRed, or luciferase, or the hexokinase-1 gene under the regulation of either CMV or RIP promoters were incorporated into cationic liposomes, which were then attached to microbubbles containing perfluoropropane gas within a phospholipid shell. The mean diameter and concentration of the microbubbles were 1.9±0.2 μm and 5.2±0.3×10⁹ bubbles/ml, respectively. The amount of plasmid adsorbed to the microbubbles was 250±10 μg/ml. One milliliter of plasmid-microbubble solution or control (microbubbles without plasmid) was infused via the right internal jugular vein of anesthetized Sprague-Dawley rats (250 g) over 20 minutes. Ultrasound was directed at the pancreas to destroy these microbubbles within the pancreatic microcirculation; microbubble infusion without ultrasound was also used as a control.

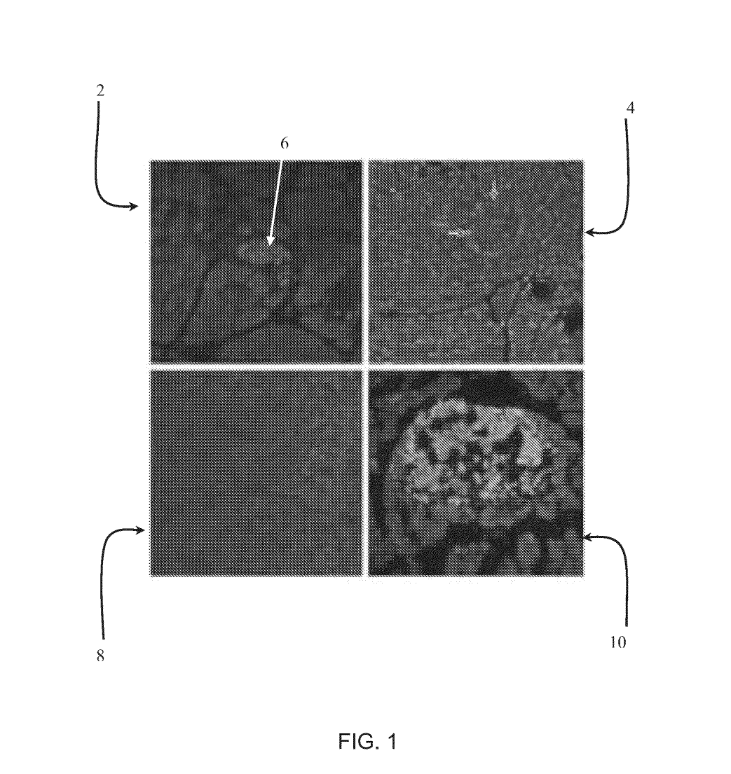

In Situ PCR for Plasmid DNA. FIG. 1 (top panel) shows the results of in situ PCR directed against plasmid DNA. Plasmid DNA is seen throughout the pancreas in a nuclear pattern, including the islets. Similar patterns of homogeneous nuclear tissue localization of the plasmid were observed in the left kidney, spleen, and portions of the liver that were within the ultrasound beam. Plasmid was not present in right kidney or skeletal muscle, organs that lie outside of the ultrasound beam. This was the case for plasmids containing either the CMV or RIP promoters, and either the LacZ or DsRed marker genes. Controls (microbubbles without plasmid or plasmid-microbubbles without ultrasound) did not show any evidence of plasmid within the pancreas. This figure demonstrates that the ultrasound treatment released the plasmid within the pancreas and its immediate vicinity.

In Situ RT-PCR for mRNA. In order to confer islet specific expression, a reporter construct driven by the rat insulin promoter (RIP) was delivered by UTMD. FIG. 1 (bottom panel) shows a representative example of in situ RT-PCR directed against the mRNA corresponding to the DsRed transcript expressed under control of the RIP promoter. DsRed mRNA is seen throughout the islets, but not in the pancreatic parenchyma, indicating that the RIP promoter directed transcription of the UTMD-delivered DsRed cDNA only in the endocrine pancreas. There was no signal detected in controls, including microbubbles without plasmid, LacZ plasmid-microbubbles, or DsRed plasmid-microbubbles without ultrasound.

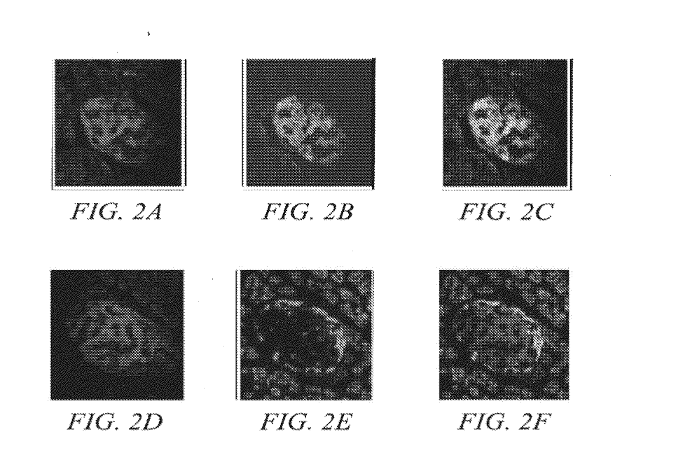

Demonstration of Specific Targeting of DsRed to Islet Beta-Cells by Confocal Microscopy. Next, the expression of DsRed protein was examined to determine if expression was confined to insulin producing beta-cells within the pancreatic islets. FIG. 2 demonstrates expression of the DsRed protein within the central core of islet cells, consistent with the known localization of beta-cells within rat islets. The DsRed protein (left panel, top) was identified with a red filter at an excitable wavelength of 568 nm and an emission wavelength of 590-610 nm. Beta-cells were identified specifically by immunohistochemical staining with a fluorescence-tagged antibody directed against insulin at an excitable wavelength of 488 nm and an emission wavelength of 490-540 nm (middle panel, top). Co-localization of the DsRed and insulin signals (right panel, top) confirms that DsRed plasmid expression was present in islet beta-cells. DsRed signal was only present in islet tissue that co-stained with anti-insulin, indicating a high degree of beta-cell specificity. In addition, there were islets identified by insulin staining that did not show DsRed expression. Examination of sections from rats infused with control microbubbles (without plasmid) or control plasmid (LacZ) did not show any detectable DsRed signal (data not shown).

The location of DsRed expression relative to glucagon-producing alpha cells is also shown in FIG. 2 (bottom panel). The DsRed protein is shown in the left bottom panel using a red filter. The alpha cells are identified on the islet periphery by immunohistochemical staining with a fluorescent antibody directed against glucagon (bright green signal, middle panel, bottom). Confocal microscopy (right panel, bottom) shows that the DsRed signal never co-localizes with the glucagon signal, which remains bright green and located on the islet periphery.

The efficiency of islet transfection was calculated by counting the number of DsRed-positive islets divided by the total number of islets (anti-insulin positive)×100. Results are shown in Table 1. Transfection efficiency was significantly higher for islets treated with the RIP-DsRed compared to CMV-DsRed plasmid (67±7% vs 20±5%, F=235.1, p<0.0001). As noted above, islets treated with control microbubbles (no plasmid or LacZ plasmid) did not show any detectable transfection.

TABLE 6
Transfection rate of islets determined as number of DsRed
positive islets/number of anti-insulin positive islets × 100.

| Rat # - plasmid | Slide 1 | Slide 2 | Slide 3 | Total |
|---|---|---|---|---|
| 1 - RIP-DsRed | 16/23 (69%) | 18/22 (81%) | 15/21 (71%) | 49/66 (74%) |
| 2 - RIP-DsRed | 19/32 (59%) | 17/27 (63%) | 15/22 (68%) | 51/81 (63%) |
| 3 - RIP-DsRed | 8/12 (67%) | 9/14 (64%) | 7/10 (70%) | 24/36 (67%) |
| 4 - RIP-DsRed | 17/32 (53%) | 20/29 (69%) | 18/28 (64%) | 55/89 (62%) |
| 5 - CMV- | 3/21 (14%) | 7/25 (28%) | 2/12 (17%) | 12/61 (19%) |
| DsRed | | | | |
| 6 - CMV- | 9/32 (28%) | 7/30 (23%) | 6/31 (19%) | 22/93 (24%) |
| DsRed | | | | |
| 7 - CMV- | 4/20 (20%) | 4/17 (24%) | 5/19 (26%) | 13/56 (23%) |
| DsRed | | | | |
| 8 - CMV- | 6/35 (17%) | 4/30 (14%) | 4/28 (15%) | 14/93 (15%) |
| DsRed | | | | |
| 9 - Control | 0/24 | 0/32 | 0/30 | 0/86 |

Taken together, these data demonstrate that coupling of UTMD with plasmids in which transgene expression is controlled by RIP results in efficient delivery of genes in a highly targeted, if not exclusive fashion to islet β-cells in living rats.

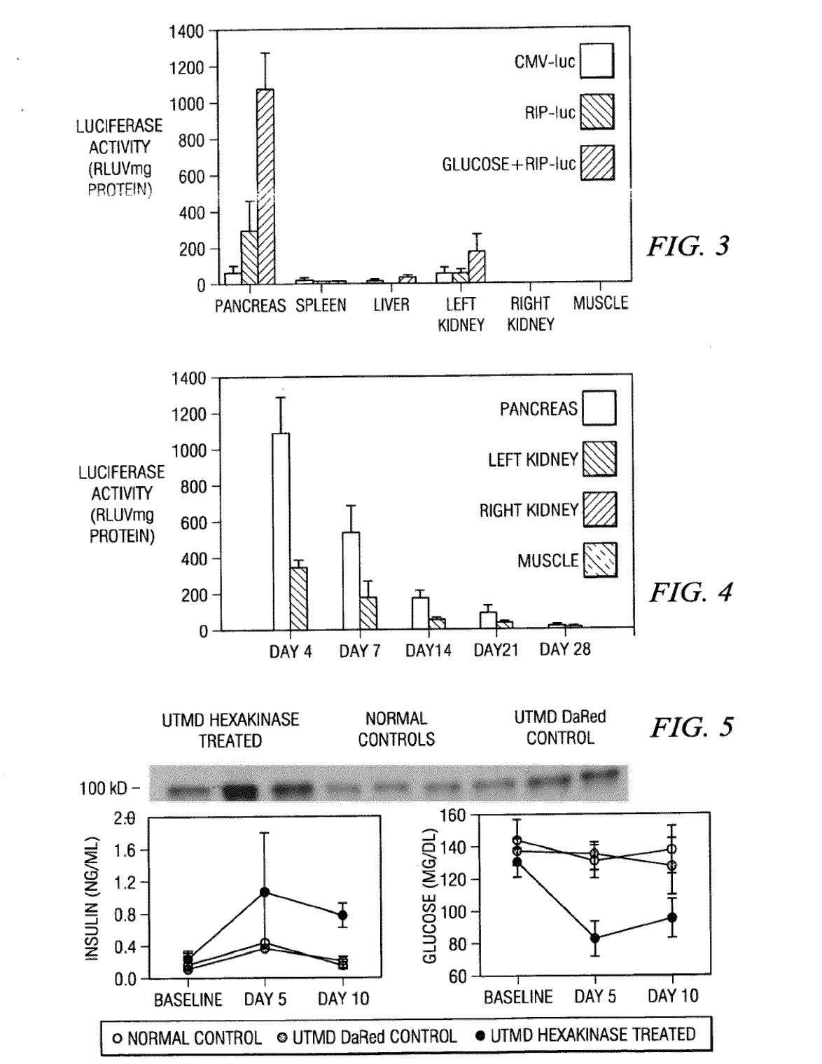

Quantitative Luciferase Gene Expression. Quantified gene expression in the pancreas was also compared to other organs within the ultrasound beam (left kidney, spleen, liver) and outside the ultrasound beam (right kidney, hindlimb skeletal muscle). Rats were sacrificed at day 4 after UTMD and luciferase activity measured in each organ and indexed for protein content as RLU/mg protein. FIG. 3 shows a comparison of luciferase activity in these organs for three groups of rats (n=3 rats per group). Three groups of rats were included in the study: animals that received CMV-luciferase microbubbles, fed on normal chow and water, animals that received RIP-luciferase microbubbles fed on normal chow and water, and animals that received RIP-luciferase microbubbles and received normal chow plus water supplemented with 20% glucose). Animals were provided these diets for 4 days prior to sacrifice. In animals that received CMV-luciferase, a low level of activity was detected in all organs within the ultrasound beam. No activity was detected in skeletal muscle or right kidney, which lie outside the ultrasound beam. By ANOVA, the difference in pancreatic luciferase activity between organs was statistically significant (F=42.4, p<0.0001), due to the markedly higher activity in pancreas compared to the other organs. Of particular importance, the RIP-luciferase plasmid increased pancreatic activity by 100-fold compared to liver (298±168 RLU/mg protein vs 2.9±0.8 RLU/mg protein), indicating that this technique obviates the problem of hepatic uptake seen with viral vectors.³

The RIP-luciferase plasmid increased pancreatic luciferase activity by 4-fold compared to CMV-luciferase (298±168 RLU/mg protein vs 68±34 RLU/mg protein, p<0.0001). Glucose feeding further increased pancreatic luciferase activity by 3.5-fold over RIP-luciferase alone (1084±192 RLU/mg protein vs 298±168 RLU/mg protein, p<0.0001), indicating that the RIP-luciferase transgene was appropriately regulated by glucose following delivery to islets by UTMD. Surprisingly, glucose feeding also caused regulation of luciferase expression in the left kidney compared to RIP-luciferase alone (172±102 RLU/mg protein vs 53±23 RLU/mg protein, p=0.0057), suggesting that the rat insulin promoter responds to glucose even when localized to the kidney. As such, the present invention may be used to provide controlled expression in more that one organ.

Time course of gene expression by UTMD. In a separate group of rats, the time course of gene expression by UTMD was measured using the RIP-luciferase plasmid. Luciferase activity was measured by sacrificing 3 rats each at 4, 7, 14, 21, and 28 days after UTMD. As shown in FIG. 4, luciferase activity drops by half from day 4 to day 7 and is nearly undetectable by day 21 (F=234, p<0.0001).

Regulation of Insulin Secretion and Circulating Glucose Levels by UTMD-mediated delivery of the Hexokinase-1 Gene. Previous studies have demonstrated that overexpression of low Km hexokinases (e.g., hexokinase I) results in a left-shift in the glucose dose response for insulin secretion, due to increased stimulus/secretion coupling at low glucose.^{3,20} Therefore, the hexokinase I gene was used to determine if gene delivery to islet β-cells by UTMD occurs with an efficiency sufficient to allow discernable changes in islet function in the context of the whole animal. Six rats were infused with microbubbles containing a plasmid with the hexokinase 1 gene under control of the RIP promoter. Controls included rats infused with RIP-DsRed-containing microbubbles (n=3) and sham-operated normal rats (n=3). Serum measurements of glucose and insulin were obtained at baseline, and at days 5 and 10 after UTMD.

As shown in FIG. 5, there was no significant change over time in serum insulin or glucose levels in the RIP-DsRed or sham surgery control groups. In contrast, serum insulin increased by 4-fold at day 5 and remained elevated at day 10 in the RIP-hexokinase I-treated groups (F=11.5, p=0.0033 by repeated measures ANOVA, treated vs controls). Correlating with the increase in insulin, serum glucose levels decreased by nearly 30% in the RIP-hexokinase I-treated rats at day 5 (F=19.8, p=0.0005 by repeated measures ANOVA, treated vs controls), and then remained low out to day 10. Further evidence of highly efficient delivery of the hexokinase I gene to pancreatic islets by UTMD is provided by immunoblot analysis of hexokinase I protein levels in islets isolated at day 10. These data show a clear increase in immunodetectable hexokinase I protein in islets of 3 rats subjected to UTMD with the RIP-hexokinase I plasmid relative to either control group. In sum, the data of FIG. 5 clearly demonstrate the use of UTMD for high efficiency gene delivery to pancreatic islet β-cells in living animals.

Safety of UTMD. Histologic sections of the pancreas did not reveal any evidence of inflammation or necrosis after UTMD. In 0.4 rats, serum amylase and lipase were measure at baseline, 1 hr, and 24 hrs after UTMD; values were normal and did not increase with UTMD. Rats subjected to UTMD gained weight normally and demonstrated no abnormal behaviors. Moreover, rats that received the RIP-DsRed plasmid experienced no significant changes in circulating glucose or insulin levels, suggesting maintenance of normal metabolic homeostasis.

This example described a novel method for efficient gene delivery to the pancreatic islets. Delivery of plasmid DNA and its subsequent expression by in situ PCR and in situ RT-PCR directed against the plasmid and its mRNA was shown. Further, gene expression in the pancreas was confined to beta-cells when UTMD was applied in conjunction with a plasmid in which RIP was used to direct transgene expression. Moreover, it was demonstrated that the RIP-luciferase plasmid retained responsiveness to physiological signals following delivery to islets via UTMD, as glucose feeding caused clear increases in reporter gene activity. Although there are examples of transgene expression in pancreatic islets of rodents achieved by microinjection of fertilized embryos,²¹⁻²⁷ this is the first example of in vivo gene delivery to pancreatic islets of living, adult animals.

The efficacy of the UTMD method for delivery of a gene was determined to show modulatation of beta-cell function. The hexokinase I gene was selected for this purpose. Pancreatic islet beta-cells normally express hexokinase IV (also known as glucokinase) as their predominant glucose phosphorylating enzyme, and the high S_{0.5} of the enzyme for glucose (approximately 6 mM) allows it to regulate the rate of glucose metabolism and control glucose-stimulated insulin secretion at physiologic glucose concentrations. Hexokinase I, in contrast, has a low S_{0.5} for glucose (approximately 0.5 mM). For comparison, it is know that Adenovirus-mediated expression of hexokinase I in rat islets results in a left-shift in glucose concentration-dependent changes in glycolysis and glucose-stimulated insulin secretion.²⁰ Moreover, expression of a low Km yeast hexokinase in beta-cells of transgenic mice was shown to cause hyperinsulinism and hypoglycemia.³ Based on these findings, the present invention was found to efficiently delivery hexokinase I to beta-cells by UTMD as demonstrated by a similar phenotype of hyperinsulinism and hypoglycemia, which was as observed and summarized in FIG. 5.

This example also described the safe and efficacious delivery of DNA constructs to beta-cells with several advantages: 1) no viral vectors are required for efficient gene transfer, limiting concerns for inflammatory responses or insertional mutagenesis;⁵ 2) use of the RIP promoter in these plasmid constructs provides a remarkable degree of beta-cell specificity within islets, with little to no expression of the DsRed reporter gene in glucagon producing alpha cells; 3) the microbubbles loaded with plasmid can be delivered via the systemic circulation, obviating the need for invasive surgery such as would be required for local delivery to pancreatic vessels; and 4) there was no evidence of pancreatic damage arising as a result of microbubble infusion and local application of ultrasound in the pancreas.

Against these very positive features of this technology is balanced one unanticipated finding. It was found that significant expression of the luciferase transgene was achieved under control of the RIP promoter in kidney, which inevitably lies in the path of the ultrasound beam when targeting the pancreas. Moreover, renal reporter gene expression was found to be responsive to glucose. An enhanced rat insulin promoter has been shown to express human growth hormone (hGH) in brain, thymus, and kidney in mice.²⁸ Insulin is known to affect expression of adenosine²⁹ and angiotensinogen³⁰ in the kidney. Using the present invention it is possible to also target the kidney for gene and drug deliver, e.g., for delivery of RIP-enhanced renal gene expression.

To reduce or avoid kidney expression a focused ultrasound transducer may be used to limit microbubble destruction to a pre-specified region of interest. In these studies, a transducer developed for clinical echocardiography, in which microbubble destruction occurred throughout the length, width, and breadth of the ultrasound beam may be used. Alternatively, it may be possible to modify or truncate the RIP promoter such that beta-cell expression is maintained in the absence of transgene expression in kidney.

The compositions and methods described in this example may be used for the treatment of both major forms of diabetes, and also represents a method of evaluating the relevance of candidate disease genes in the endocrine pancreas. Type 1 diabetes involves the autoimmune destruction of pancreatic islet beta-cells. Several approaches have been suggested for protecting beta-cells from immune-mediated destruction, including blockade of T-cell and macrophage-mediated destruction by prevention of cell/cell interactions, or, alternatively, the instillation of genes that can protect against damage caused by inflammatory cytokines or reactive oxygen species.² However, testing of these approaches has been limited to transgenic (germ-line) manipulation or ex-vivo engineering of pancreatic islets prior to transplantation. The method taught in this example provide for genetic engineering of islets in situ, such that various strategies for enhancing islet survival can be tested in animal models of type 1 diabetes in the pre-diabetic phase.

The compositions and methods taught herein may also be used for type 2 diabetes. In this disease, beta-cells appear to suffer the dual lesions of functional insufficiency and a gradual (but not complete) diminution of cell mass.³¹ The mechanisms involved in development of beta-cell dysfunction and loss of beta-cell mass in type 2 diabetes are not fully understood, but theories about the potential roles of chronic hyperlipidemia and lipid overaccumulation in beta-cells (“lipotoxicity”),^{32,33} as well as damaging effects of chronic exposure to glucose (“glucotoxicity)³⁴ have been developed. The technology taught herein allows genes that modulate lipid or glucose metabolism to be delivered to islets in models of type 2 diabetes. Moreover, the group of diseases known as Maturity Onset Diabetes of the Young (MODY) appear to include+a set of single gene mutations involving transcription factors or metabolic enzymes that control beta-cell function.³⁵ The present invention allows a rapid method to test beta-cell candidate genes that emerge from human genetic studies in the context of adult animals. Finally, with the advent of technologies for suppression of gene expression such as small interference RNAs (siRNAs) and their application to pancreatic islets,^{36,37} UTMD-mediated delivery of siRNA-containing plasmids may be used for control (upregulation, downregulation) of specific genes in beta-cell function and survival in living animals.

Rat UTMD Protocol. Sprague-Dawley rats (250-350 g) were anesthetized with intraperitoneal ketamine (100 mg/kg) and xylazine (5 mg/kg). A polyethylene tube (PE 50, Becton Dickinson, MD) was inserted into the right internal jugular vein by cutdown. The anterior abdomen was shaved and an S3 probe (Sonos 5500, Philips Ultrasound, Andover, Mass.) placed to image the left kidney and spleen, which are easily identified. The pancreas lies between them, so the probe was adjusted to target the pancreas and clamped in place. One ml of microbubble solution was infused at a constant rate of 3 ml/h for 20 minutes using an infusion pump. Throughout the duration of the infusion, microbubble destruction was achieved using ultraharmonic mode (transmit 1.3 MHz/receive 3.6 MHz) with a mechanical index of 1.2-1.4 and a depth of 4 cm. The ultrasound pulses were ECG-triggered (at 80 ms after the peak of the R wave) to deliver a burst of 4 frames of ultrasound every 4 cardiac cycles. These settings have previously been shown to be the optimal ultrasound parameters for gene delivery using UTMD.⁵ At the end of each study the jugular vein was tied off and the skin closed. All rats were monitored after the experiment for normal behavior. Rats were sacrificed 4 days later and the pancreas were harvested.

Manufacture of Plasmid-Containing Lipid-Stabilized Microbubbles. Certain lipid-stabilized microbubbles were prepared as previously described by the present inventors.^{5,6} In the present invention, a solution of DPPC (1,2-dipalmitoyl-sn-glycero-3-phosphatidylcholine, Sigma, St. Louis, Mo.) 2.5 mg/ml; DPPE (1,2-dipalmitoyl-sn-glycero-3-phosphatidylethanolamine, Sigma, St. Louis, Mo.) 0.5 mg/ml; and 10% glycerol was mixed with 2 mg of plasmid solution in a 2:1 ratio. Aliquots of 0.5 ml of this phospholipid-plasmid solution were placed in 1.5 ml clear vials; the remaining headspace was filled with the perfluoropropane gas (Air Products, Inc, Allentown, Pa.). Each vial was incubated at 40° C. for 30 min and then mechanically shaken for 20 seconds by a dental amalgamator (Vialmix™, Bristol-Myers Squibb Medical Imaging, N. Billerica, Mass.). The lipid-stabilized microbubbles appear as a milky white suspension floating on the top of a layer of liquid containing unattached plasmid DNA. The subnatant was discarded and the microbubbles washed three times with PBS to removed unattached plasmid DNA. The mean diameter and concentration of the microbubbles in the upper layer were measured by a particle counter (Beckman Coulter Multisizer III).

Plasmid Constructs. Rat genomic DNA was extracted from rat peripheral blood with a QIAamp Blood kit (Qiagen Inc, Valencia, Calif.) according to the manufacturer's instructions. A DNA fragment containing the rat insulin I promoter (RIP), exon 1, intron 1 (only intron) and 3 bp (GTC) of 5′ end of exon 2 ((from −412 to +165) was PCR amplified from Sprague-Dawley Rat DNA by using the following PCR primers that contain a restriction site at the 5′ ends (the restriction sites are underlined):

primer 1 (XhoI)
(SEQ ID NO.: 1)
5'-CAACTCGAGGCTGAGCTAAGAATCCAG-3';
primer 2 (EcoRI)
(SEQ ID NO.: 2)
5'-GCAGAATTCCTGCTTGCTGATGGTCTA-3'.

The corresponding PCR products were verified by agarose gel electrophoresis and purified by QIAquick Gel Extraction kit (QIAGEN). To confirm the sequences, direct sequencing of PCR products was performed with dRhodamine Terminator Cycle Sequencing Kit (PE Applied Biosystems, Foster City, Calif.) on an ABI 3100 Genomic Analyzer. The PCR amplified fragments were digested with XhoI and EcoRI and then ligated into the XhoI-EcoRI sites of pDsRed-Express-1, a promoterless Discosoma sp. red fluorescent protein (DsRed) plasmid (BD. Biosciences). Ligation reactions were carried out in 20 μl of 20 mM Tris-HCL, 0.5 mMATP, 2 mM dithiothreitol and 1 unit of T4 DNA ligase. Cloning, isolation and purification of this plasmid were performed by standard procedures, and once again sequenced to confirm that no artifactual mutations were present.

Plasmid expressing the hexokinase 1 gene under the RIP promoter was made as following: Total mRNA was extracted from a Sprague-Dawley rat pancreas with a QIAamp kit (Qiagen Inc, Valencia, Calif.) according to the manufacturer's instructions. And then mRNA was reversed into cDNA with a SuperScript first-strand synthesis system for RT-PCR kit (Invitrogen). A full length cDNA of the hexokinase 1 cDNA was PCR amplified by using the following PCR primers that contain a restriction site at the 5′ ends (the restriction sites are underlined):

primer 1 (EcoRI)
(SEQ ID NO.: 3)
5'-AAAGAATTCATGATCGCCGCGCAACTACTGGCCTAT-3';
primer 2 (Not I)
(SEQ ID NO.: 4)
5'-AAAGCGGCCGCTTAGGCGATCGAAGGGTCTCCTCT-3'

The product was confirmed by sequencing. The DNA was digested with EcoR1 and NotI and then ligated into the corresponding sites of pRIP3.1 vector. Cloning, isolation and purification of this plasmid were performed by standard procedures, and once again sequenced to confirm that no artifactual mutations were present.

In Situ-PCR for Detection of DsRed DNA. DsRed Primers. A single pair of DsRed primers were used directed against the DsRed DNA; they are DsRed 125⁺ (5′-GAGTTCATGCGCTTCAAGGTG-3′) (SEQ ID NO.:5) and DsRed 690⁻ (5′-TTGGAGTCCACGTAGTAGTAG-3′) (SEQ ID NO.:6).

Immediately after sacrifice, blood was removed from the rats by 200 ml intra-arterial cooled saline followed by perfusion fixation with 100 ml of 2% paraformaldehyde and 0.4% glutaraldehyde. The pancreas was cut into 0.5 cm pieces and placed into 20% sucrose solution overnight in 4° C. and then put into OTC molds at −86° C. Frozen sections 5 μm in thickness were placed on silane coated slides and fixed in 4% paraformaldehyde for 15 min at 4° C., quenched with 10 mM glycine in PBS for 5 minutes, rinsed with PBS, permeabilized with 0.5% Triton X-100 in PBS for 10 min, and rinsed with PBS for 10 min. A PCR DIG Prob Synthesis Kit (Roche Co.; Cat. NO: 1636090) was used. A coverslip was anchored with a drop of nail polish at one side. The slide was then placed in aluminum ‘boat’ directly on the block of the thermocycler. A 50 μl PCR reaction solution (0.8 units of Taq DNA polymerase, 2 μl of DsRed primers, 3 μl of DIG-dNTP, 5 μl of 10× buffer and 40 μl of water) was added to each slide and covered by the AmpliCover Disc and Clips using the Assembly Tool (Perkin Elmer) according to the manufacturer's instructions. In situ PCR was performed using Perkin-Elmer GeneAmp system 1000 as follows: after an initial hold at 94° C. (1 min), the PCR was carried out for 11 cycles (94° C. for 1 min, 54° C. for 1 min, and 72° C. for 2 min). After amplification, the slide was immersed 2×SSC for 10 min and 0.5% paraformaldehyde for 5 min and PBS for 5 min 2 times. The digoxigenin incorporated-DNA fragment was detected using a fluorescent antibody enhancer set for DIG detection (Roche) followed by histochemical staining. First, the sections were incubated with blocking solution for 30 min to decrease the non-specific binding of the antibody to pancreas tissue. Then, the sections were incubated with 50 μl of anti-DIG solution (1:25) for 1 h at 37° C. in a moisturized chamber. Then the slides were washed with PBS three times with shaking, each for 5 min. again the slides were incubated with 50 μl of anti-mouse-1gG-digoxigenin antibody solution (1:25) for 1 hr at 37° C. The slides were washed with PBS three times with shaking, each for 5 min again. The slides were incubated with 50 μl of anti-DIG-fluorescence solution (1:25) for 1 hr at 37° C. The slides were washed with PBS three times with shaking, each for 5 min again. Finally, the sections were dehydrated in 70% EtOH, 95% EtOH and 100% EtOH, each for 2 min, cleared in xylene and coverslipped.

In Situ RT-PCR for Detection of DsRed mRNA. DsRed primers. A single pair of DsRed primers were used directed against the DsRed cDNA, they are DsRed 125⁺ (5′-GAGTTCATGCGCTTCAAGGTG-3′) (SEQ ID NO.:7) and DsRed 690⁻ (5′-TTGGAGTCCACGTAGTAGTAG-3′) (SEQ ID NO.:8).

Perfusion fixed frozen sections were prepared as described above. DNase treatment was performed with 50 μl of cocktail solution (Invitrogen) (5 μl of DNase I, 5 μl of 10× DNase buffer, and 40 μl of water) on each slide, coverslipped, incubated at 25° C. overnight, and then washed with PBS 5 min 2 times.

Reverse transcription: First-strand cDNA synthesis was performed on each slide in a 50 μl total volume with 50 μl of cocktail solution (Superscript First-strand synthesis system for RT-PCR, Invitrogen kit #11904-018) (1 μl of DsRed727⁻ primers (5′-GATGGTGATGTCCTCGTTGTG-3′) (SEQ ID NO.:9), 5 μl of DTT solution, 2.5 μl of dNTP, 5 μl of 10× buffer, 5 μl of 25 mM MgCl, 29 μl of water and 2.5 μl of SuperScript II RT). A coverslip was placed and the slides incubated at 42° C. for 2 hrs; washed with PBS 5 min 2 times, rinsed with 100% ETOH for 1 min and dried.

Immunohistochemistry for Detection of DsRed protein, Insulin, and Glucagon. Cryostat sections 5 μm in thickness were fixed in 4% paraformaldehyde for 15 min at 4° C. and quenched for 5 min with 10 mM glycine in PBS. Sections were then rinsed in PBS 3 times, and permeabilized with 0.5% Triton X-100 in PBS for 10 min. Sections were blocked with 10% goat serum at 37° C. for 1 hr and washed with PBS 3 times. The primary antibody (Sigma Co.) (1:50 dilution in block solution) was added and incubated at 4° C. overnight. After washing with PBS three times for 5 min, the secondary antibody (Sigma Co., anti-mouse 1gG conjugated with FITC) (1:50 dilution in block solution) was added and incubated for 1 hr at 37° C. Sections were rinsed with PBS for 10 min, 5 times, and then mounted.

Luciferase Assay. To quantitate expression of the luciferase transgene, the pancreas, both kidneys, spleen and skeletal muscle were pulverized in a Polytron and incubated with luciferase lysis buffer (Promega Co,), 0.1% NP-40, and 0.5% deoxycholate and proteinase inhibitors. The resulting homogenate was centrifuged at 10,000 g for 10 minutes and 100 μl of luciferase reaction buffer (Promega) was added to 20 μl of the clear supernatant. Light emission was measured by a luminometer (TD-20/20, Turner Designs Co.) in RLU (relative light units). Total protein content was determined by the Lowry method (BCA protein assay reagent, Pierce Co.) from an aliquot of each sample. Luciferase activity was expressed as RLU/mg protein.

Hexokinase I Western Blot. Sections of whole pancreas were harvested at sacrifice (day 10 after UTMD gene delivery) from each rat and homogenized in Tris buffer. Equal amounts of protein from these tissue homogenates were subjected to electrophoresis using a 12% BioRad gel, blocked, and incubated with mouse anti-hexokinase I antibody. Immunoreactive bands were visualized with chemiluminescent substrate (ECL, Amersham, Piscataway, N.J., USA).

Statistical Analysis. Differences in luciferase activity between experimental groups were compared by two-way ANOVA. Repeated measures ANOVA was used to evaluate the results of the time course experiment. Two-way repeated measures ANOVA was used to assess the temporal change in serum insulin and glucose between hexokinase 1-treated rats and control groups. A p value <0.05 was considered statistically significant. Post-hoc Scheffe tests were performed only when the ANOVA F values were statistically significant.

### Example 10

Targeting of VEGF-mediated angiogenesis to rat myocardium using ultrasonic destruction of microbubbles. Myocardial angiogenesis mediated by human VEGF₁₆₅ cDNA was promoted in rat myocardium using an in vivo targeted gene delivery system known as ultrasound targeted microbubble destruction (UTMD). Microbubbles carrying plasmids encoding hVEGF₁₆₅, or control solutions were infused i.v. during ultrasonic destruction of the microbubbles within the myocardium. Biochemical and histological assessment of gene expression and angiogenesis were performed 5, 10 and 30 days after UTMD. UTMD-treated myocardium contained hVEGF₁₆₅ protein and mRNA. The myocardium of UTMD-treated animals showed hypercellular foci associated with hVEGF₁₆₅ expression and endothelial cell markers. Capillary density in UTMD-treated increased 18% at 5 days and 33% at 10 days, returning to control levels at 30 days (p<0.0001). Similarly, arteriolar density increased 22% at 5 days, 86% at 10 days, and 31% at 30 days (p<0.0001). Thus, non-invasive delivery of hVEGF₁₆₅ to rat myocardium by UTMD resulted in significant increases in myocardial capillary and arteriolar density.

The stimulation of new blood vessel growth by vascular growth factors and/or the genes that express them has long been proposed as a potential treatment for myocardial ischemia.³⁸⁻⁴¹ Although great strides have been made in understanding angiogenesis (development of new capillaries) and arteriogenesis (development of larger vessels containing an intima, media, and adventitia), translation of the basic science into clinically useful therapies has not yet occurred. It has been pointed out that the largely disappointing results of recent clinical trials of angiogenic therapies,⁴²⁻⁵² which may be explained by a variety of factors, including patient selection, incomplete understanding of the optimal angiogenic agent or combination of agents, a limited time course of treatment (usually a single fixed dose), and suboptimal delivery techniques. The latter two issues are interrelated in that current methods of delivering angiogenic agents are limited to direct myocardial injection or intracoronary infusion, invasive techniques that are not well suited for repeated treatments.

This example demonstrates a non-invasive method, ultrasound targeted microbubble destruction (UTMD), which allows specific targeting of gene therapy to the heart. Briefly, cationic liposomes containing plasmid DNA are attached to the phospholipid shell of gas-filled microbubbles 2-4 μm in diameter. These microbubble-liposome complexes are infused intravenously and destroyed within the myocardial microcirculation by low frequency ultrasound. As shown hereinabove, UTMD can be used to deliver reporter genes selectively to the pancrease and kidney. Other examples have shown delivery to the heart;⁵³⁻⁵⁵ however, there have been no reports of its use to achieve a biological effect. UTMD was used to promote angiogenesis by non-invasive delivery of the human vascular endothelial growth factor 165 (hVEGF₁₆₅) expression construct to rat myocardium.

Male Sprague-Dawley rats underwent rats UTMD treatment with microbubbles containing a plasmid encoding the hVEGF₁₆₅ gene, or three different controls, hVEGF₁₆₅ plasmid without microbubbles, microbubbles alone without plasmid, or saline. All rats tolerated the UTMD procedure without complications and survived to their designated sacrifice at either 5, 10, or 30 days after the procedure. Left ventricular mass and fractional area shortening showed no significant difference between UTMD-treated or control rats (table 7), suggesting that left ventricular hypertrophy or systolic dysfunction did not occur as a result of UTMD. At sacrifice, animals exhibited no changes in activity or feeding, and lacked any evidence of edema, hemangioma or other tumors.

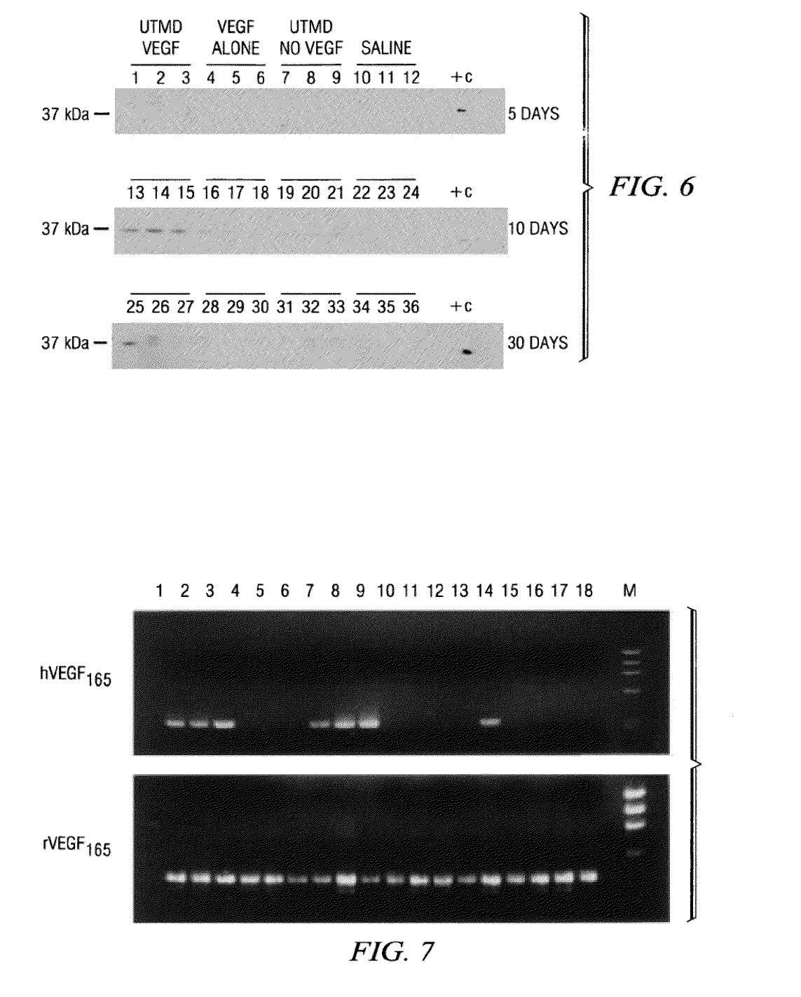

Presence of hVEGF₁₆₅ in Rat Myocardium. Immunoblotting revealed a prominent 37 kDa band consistent with hVEGF₁₆₅ in homogenates of cardiac tissue 10 days after treatment (FIG. 6). Faint bands, probably representing endogenous VEGF, were seen in control animals. Increases in hVEGF₁₆₅ protein were restricted to the tissue targeted by UTMD. Homogenates of organs that lie adjacent to, but outside of the region ultrasound targeting, such as liver, lung and spleen, showed no similar increase in hVEGF₁₆₅ protein. These findings confirm tissue specificity of the exogeneous angiogenic gene that was restricted to the insonified region. hVEGF₁₆₅ was not detected in any control animals.

TABLE 7
Left ventricular (LV) fractional shortening
and mass in treated vs control animals.

| | LV fractional | | | | | |
|---|---|---|---|---|---|---|
| | shortening (%) | | | LV mass (g) | | |
| | UTMD | | | UTMD | | |
| Time | VEGF | Control | | VEGF | Control | |
| point | Group | Groups | p | Group | Groups | p |
| Day 0 | 53.8 ± 3.0 | 59.6 ± 2.8 | 0.26 | 4.17 ± 0.1 | 3.82 ± 0.1 | 0.14 |
| (Prior to | | | | | | |
| UTMD) | | | | | | |
| Day 5 | 63.9 ± 0.8 | 66.8 ± 3.6 | 0.39 | 4.08 ± 0.2 | 3.85 ± 0.1 | 0.34 |
| after | | | | | | |
| UTMD | | | | | | |
| Day 10 | 60.7 ± 4.1 | 64.0 ± 8.5 | 0.77 | 4.54 ± 0.3 | 4.22 ± 0.1 | 0.44 |
| after | | | | | | |
| UTMD | | | | | | |
| Day 30 | 59.4 ± 5.0 | 67.9 ± 2.1 | 0.26 | 4.41 ± 0.2 | 3.93 ± 0.2 | 0.2 |
| after | | | | | | |
| UTMD | | | | | | |

Consistent with the results of Western blots, RT-PCR revealed expression of hVEGF₁₆₅ in day 5 and day 10 groups as well as one rat in day 30 group (FIG. 7), but not in control groups. To avoid any cross contamination, no PCR positive control was used for hVEGF₁₆₅. Human VEGF₁₆₅ RT-PCR products were confirmed by sequencing (data not shown).

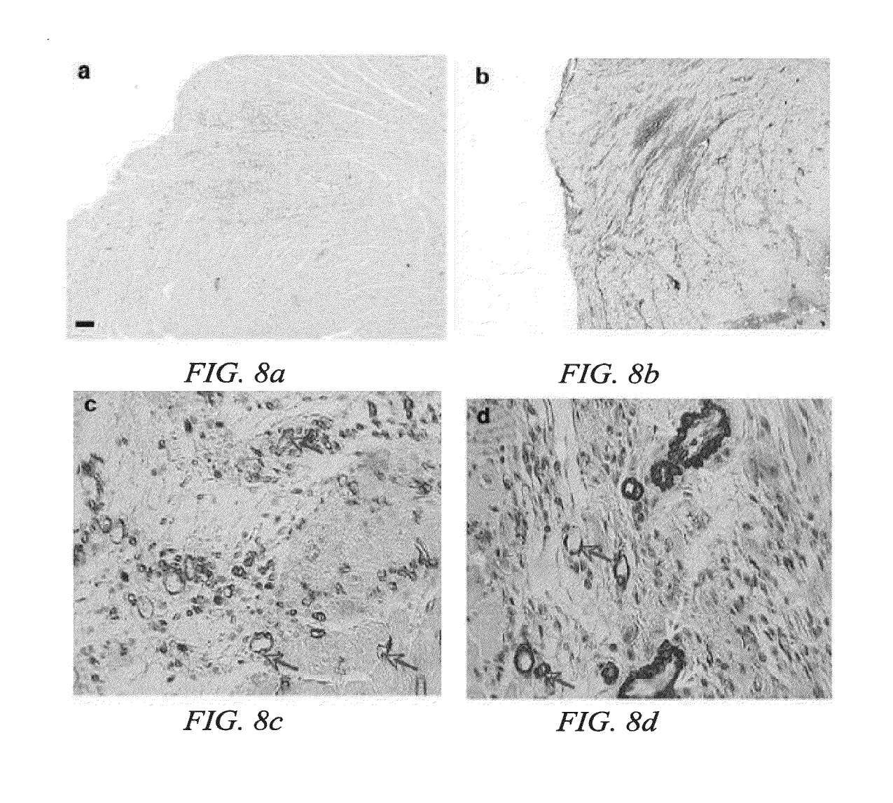

At 10 days post-treatment, histology revealed hypercellular foci in the myocardium of UTMD treated animals (FIG. 8), but not in control animals. These hypercellular foci showed staining with anti-VEGF antibody, confirming successful transfer and expression of the exogenous angiogenic gene. In addition, these foci showed staining with the endothelial cell specific markers, CD-31 and BS-I lectin. Endothelial cells in these regions displayed prominent nuclei and occasional mitotic figures. Smooth muscle α-actin staining showed pericytes covering the vessels, which is further evidence for angiogenesis. Neutrophils, monocytes, plasma cells and lymphocytes were distinctly rare and there was no myocyte necrosis. However, there was fibroblast proliferation with disorganization of the myofibrillar architecture, consistent with mild inflammation. By day 30, these foci exhibited resolution of the inflammation. None of these hypercellular foci were present in any control animal.

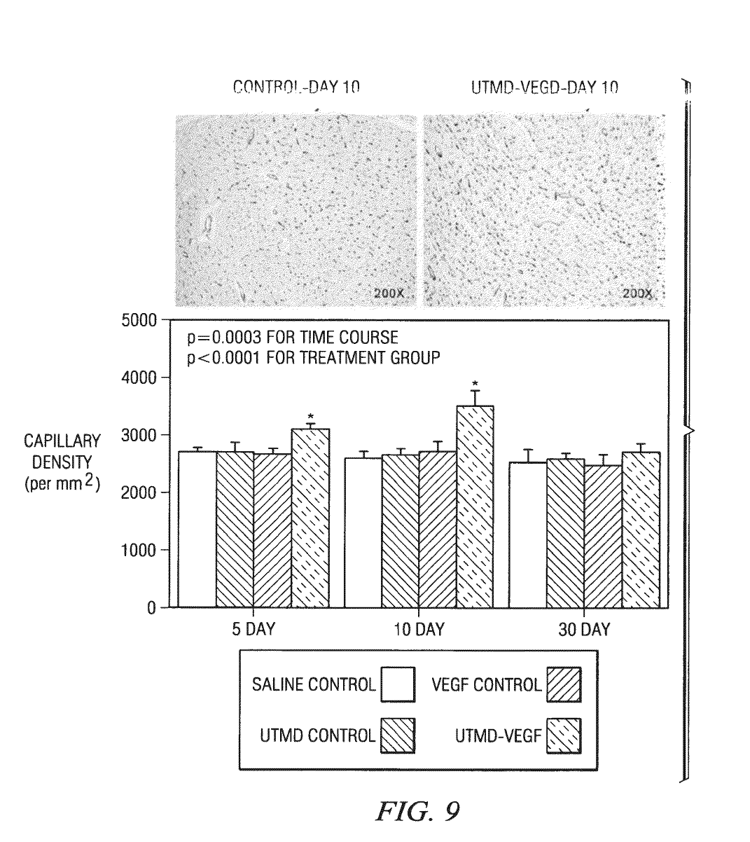

Myocardial capillary density was assessed histologically using BS-1 lectin staining (FIG. 9 top panels). Capillary density was remarkably similar in the three control groups over all 3 time periods, averaging 2606±150/mm² (FIG. 9, bottom panel). In the UTMD-treated rats, capillary density was increased by 18% at day 5 (3079±86/mm²) and 33% at day 10 (3465±283 capillaries/mm²), but returned to control levels at day 30 (2683±145/mm²). By ANOVA, the change in capillary density between treatment groups was statistically significant (F=19.25, p<0.0001).

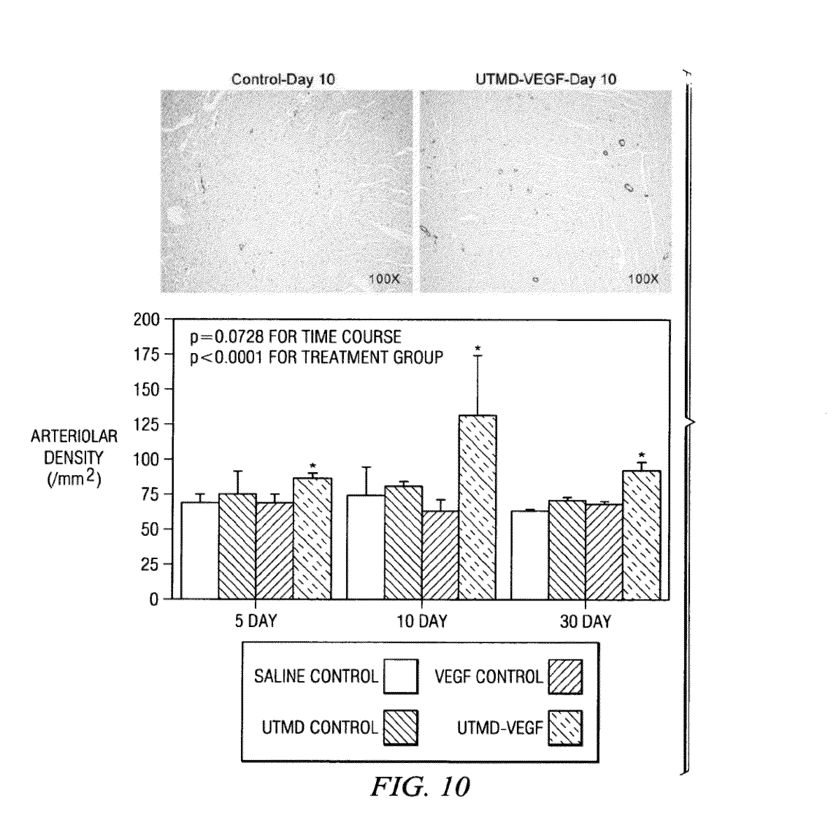

Arteriolar density was assessed using smooth muscle α-actin (sm-α-actin) staining (FIG. 10 top panels). Arteriolar density was not significantly different between control groups at the three time points studied, averaging 71±10/mm² (FIG. 10, bottom panel). In UTMD-treated rats, arteriolar density was increased by 23% at day 5 (87±3/mm²), 86% at day 10 (132±43/mm²), and 31% at day 30 (93±7/mm²). By ANOVA the change in arteriolar density between treatment groups was statistically significant (F=11.05, p<0.0001).

This example demonstrated that an angiogenic gene can be targeted non-invasively to the heart, and modify the myocardial microvasculature. Specifically, there was a transient elevation in capillary density and a more sustained elevation in arteriolar density. Notably, this is the first evidence that non-invasive delivery of a transgene to the heart has therapeutic potential in that it results in both gene expression and biological changes in the myocardium.

Increased capillary and arteriolar density was demonstrated within the myocardium after hVEGF₁₆₅ plasmid gene transfer. Limited plasmid expression (hVEGF₁₆₅ protein was detectable in all treated rats only at day 10 after gene delivery) increased both capillary and arteriolar density at 10 days of treatment. However, by day 30, regression of capillary to the baseline level was observed. Perhaps this is due to the transient nature of the plasmid expression or subsequent capillary derecruitment in the setting of normal rather than ischemic myocardium.

Arterioles also decreased from their peak at day 10 by day 30 post-treatment. However, the 30-day arteriolar density was still significantly higher than controls, indicating sustained arteriogenesis after hVEGF₁₆₅ therapy. This is an important new finding that may be related to the longer expression of hVEGF₁₆₅ after UTMD than with direct injection or intracoronary infusion. In a murine model of conditional switching of VEGF, brief exposure to VEGF causes transient growth of vessels that disappear after VEGF withdrawal.⁵⁶ In contrast, 10-14 days of VEGF stimulation produced an arteriogenic response in which mature vessels did not resorb.⁵⁶ In this example, UTMD resulted in readily detectable hVEGF₁₆₅ protein by Western blots in the rat myocardium 10 days after treatment. The prolonged duration of hVEGF₁₆₅ expression with UTMD may also facilitate the previously described protective effect of smooth muscle cell-endothelial cell interactions on the newly formed microcirculation and its important role in the vascular remodeling.⁵⁷⁻⁶⁰

An increase in capillary or arteriolar density was neither observed with either hVEGF₁₆₅ plasmid alone, nor with microbubble destruction alone. It is not surprising that i.v. VEGF does not promote angiogenesis because of the effects of circulating DNases and the absence of a mechanism for the circulating plasmid to cross the endothelial barrier. However, Song et al⁶¹ demonstrated arteriogenesis in rat skeletal muscle exposed to the low frequency ultrasound after intravenous injection of albumin microbubbles, suggesting that microbubble destruction may contribute to vascular remodeling. Ultrasonic microbubble destruction is known to cause cavitation, thermal effects, micro streaming, and free radical production, factors that could potentially interact with endothelial cells leading to their activation.⁶²⁻⁶⁵ Also, mechanical destruction of the microbubbles within the microvasculature creates capillary ruptures;^{66,67} healing of these rupture sites may have contributed to some aspect of arteriogenesis in their model. The absence of an angiogenic effect of UTMD alone in this study could be due to different responses to microbubble destruction in the myocardium compared to skeletal muscle, differences between albumin and lipid microbubble shells, or other unknown experimental variables.

The mild inflammation and disruption of myocellular architecture noted in the UTMD group is likely a result of VEGF-mediated angiogenesis by UTMD. VEGF is known to promote inflammation via several mechanisms, including endothelial cell adhesion markers, matrix metalloproteinases, and alpha-defensins.⁶⁸⁻⁷⁰ The absence of these histologic findings in the control groups indicates that simple destruction of the microbubbles alone was not sufficient to cause inflammation, nor was infusion of VEGF plasmid alone without microbubble carriers. However, it is possible that combination of VEGF plasmid and microbubble destruction are synergistic in producing an inflammatory response. It is important to note that this inflammatory response did not result in left ventricular hypertrophy or systolic dysfunction, confirming results of from the inventors' previous study on the lack of significant bioeffects of microbubble destruction in the heart.⁷¹ This example also shows that microbubble destruction, at a similar microbubble concentration and sonographic power used here, does not induce cardiac gene expression in vivo.⁷² Finally, in previous studies using reporter genes delivered to the heart by UTMD, we did not find any evidence of inflammation by histology⁷¹ or gene expression.⁷²

UTMD delivery of an hVEGF₁₆₅ expression construct was used to stimulate capillary and arteriolar growth in normal myocardium. Due to the requirement for histological evaluation, the effects of UTMD were only studies on blood vessel growth at three specific time points—days 5, 10, and 30. The establishment of timing or maximal amount of transgene expression may be determined by the skilled artisan using the compositions and methods taught herein without undue experimentation, as can the maximal amount of capillary or arteriolar response at intermediate time points. Similarly, longer time frames, e.g., after 30 days, may be observed to determined if arteriolar density returns to the baseline level or whether hypoxic conditions could sustain the arteriogenesis process as described by Hershey, et al., in rabbit hindlimb ischemia model.⁷² The expression of a reporter construct in the heart can be prolonged by repeated application of UTMD.⁷⁵ Arteriogenesis can be caused by growth of pre-existing small capillaries⁷⁴ or de novo formation of new arterioles.⁷⁵

There are a number of other known angiogenic factors, such as fibroblast growth factors (FGF), platelet-derived growth factors (PDGF), angiopoetin-2, or hypoxia-inducible factor 1-α (HIF-1α),⁷⁶ that could produce superior angiogenic or arteriogenic responses with UTMD, perhaps without some of the inflammatory consequences of VGEF. It should also be noted that angiogenesis in the vasa vasorum might promote or facilitate atherosclerosis,⁷⁷⁻⁸¹ a potential adverse effect of VEGF-gene therapy that was not addressed in this study.

UTMD may be used to deliver successfully genes to the hearts of larger mammals, e.g., humans, monkeys, dogs or pigs. The small size of the rats may make them more suitable for UTMD because the heart is small enough to be fully encompassed by the width of the ultrasound beam and because there is less tissue attenuation or lung interference.

Ultrasound targeted microbubble destruction (UTMD) directs hVEGF₁₆₅ expression to rat myocardium, with resultant increases in both capillary and arteriolar density. This method is non-invasive and allows specific targeting of gene expression to the heart and other organs. It also appears to be safe with no detrimental effect on LV function. The exact molecular mechanism of myocardial transfection by UTMD remains to be determined.

Animal preparation and gene delivery. Animal studies were performed in accord with NIH recommendations and the approval of the institutional animal research committee. Male Sprague Dawley rats (200 to 250 g, Harlan) were anesthetized with intraperitoneal ketamine (60 mg/kg) and xylazine (5 mg/kg). Hair was shaved from the precordium and neck, and a polyethylene tube (PE 50, Becton Dickinson, MD) was inserted into the right internal jugular vein by cut-down. Rats received one of four treatments: microbubbles loaded with plasmids encoding the hVEGF₁₆₅ gene under an enhanced CMV promoter (0.6 mg DNA/kg), these same plasmids (0.6 mg/kg) unattached to microbubbles, microbubbles alone without attached plasmids, or normal saline. Animals that received bubble solutions had 0.5 ml bubbles mixed with 0.5 ml of PBS infused over 20 minutes via pump (Genie, Kent Scientific). One ml of the non-bubble plasmid or saline solution was similarly infused undiluted for a total of 1 ml over 20 minutes.

During the infusion, ultrasound was directed to the heart using a commercially available ultrasound transducer (S3, Sonos 5500, Philips Ultrasound, Bothell, Wash.). A mid-ventricular, short axis view of the heart was obtained and after optimization of the image plane, the probe was clamped in place. Ultrasound was then applied in ultraharmonic mode (transmit 1.3 MHz/receive 3.6 MHz) at a mechanical index of 1.6. Four bursts of ultrasound were triggered to every fourth end-systole by ECG using a delay of 45-70 ms after the peak of the R wave. These settings have shown to be optimal for plasmid delivery by UTMD using this instrument.⁵⁴ Bubble destruction was visually apparent in all rats. The echo-contrast signal was visually absent in myocardium by the fourth pulsation. After UTMD, the jugular vein was tied off, the skin closed, and the animals allowed to recover. Animals were sacrificed at day 5 (n=12), day 10 (n=12), or day 30 (n=12) after UTMD using an overdose of sodium pentobarbital (120 mg/kg). These time points were chosen based on the inventors' previous findings of reporter gene expression after UTMD.^{54,55} Heart, lung, liver, spleen and kidney were harvested for histology and assessment of hVEGF₁₆₅ protein by Western blot and mRNA by RT-PCR.

Immunohistochemistry. The harvested tissues were fixed in methyl carnosyl and then 70% ethanol and embedded in paraffin. Five μm sections were obtained, deparaffinized, and subjected to antigen retrieval for CD31, hVEGF₁₆₅, and smooth muscle α-actin by microwave heating for 20 minutes at 900 W in 0.01 M sodium citrate, pH 6.0. Sections were blocked with 10% goat serum and endogenous peroxidase activity was quenched with 0.3% H₂O₂ in methanol. Sections were incubated with primary monoclonal antibodies according to the manufacturers recommendations: anti-CD31 at a 1:50 dilution, anti-smooth muscle α-actin at a 1:20 dilution, and anti-human VEGF-165 at 1:100 dilution, followed by biotinylated secondary antibodies: anti-mouse IgG for CD31 and smooth muscle α-actin and anti-goat IgG for VEGF. Lectin stains performed with Griffonia simplicifolia agglutinin I: BS-I lectin biotinylated antibody (Sigma-Aldrich, St Louis, Mo., USA) without antigen retrieval after blocking with 10% goat serum and quenching as above. All stains were developed with HRP-streptavidin followed by DAB chromogen and counterstained with hematoxylin.

RT-PCR. Total RNA was prepared from the specimens using an RNeasy Mini Kit (QIAGEN) according to the manufacturer's instructions. cDNA synthesis was carried out in a total 20 μl reaction with 30 ng of total RNA using a Sensiscript RT Kit (QIAGEN). PCR was performed for all samples using a GeneAmp PCR System 9700 (PE ABI) in 50 μl volume containing 2 μl cDNA, 25 μl of HotStarTaq Master Mix (QIAGEN) and 20 μmol of each primer: 5′GGAGGAGGGCAGAATCATCAC 3′ (sense) (SEQ ID NO.:10); 5′ CGCTCTGAGCAAGGCCCACAGG 3′ (antisense) (SEQ ID NO.:11). under the following conditions: an initial heating to 94° C. for 10 min, then 94° C. for 20 s, 56° C. for 20 s, 72° C. for 30 s for 48 cycles, and then at 72° C. for 5 min. The RT-PCR products were then analyzed on 2% agarose gels. A PCR reaction using rat. VEGF primers 5′ ACAGAAGGGGAGCAGAAAGCCCAT 3′ (sense primer) (SEQ ID NO.:12); 5′ CGCTCTGACCAAGGCTCACAGT 3′ (antisense primer) (SEQ ID NO.:13) served as a positive control.

VEGF Western Blot. Equal amounts of protein from tissue homogenates harvested at each time point (5, 10 and 30 days) after gene delivery, were subjected to electrophoresis through a 12% SDS polyacrylamide gel and transferred to a polyvinylidene fluoride membrane (Immobilon, Millipore, Billerica, Mass., USA), blocked, and incubated with anti-human-VEGF antibody. Immunoreactive bands were visualized with chemiluminescent substrate (ECL, Amersham, Piscataway, N.J., USA).

Capillary and arteriolar density measurement. BS-I lectin positive vessels with a diameter <10 μm and smooth muscle α-actin positive vessels with a diameter >30 μm visualized by immunohistochemistry were considered as capillaries and arterioles, respectively. Capillaries were counted by the use of light microscopy at a magnification of 400×. Five photomicrographs were taken from each slide and a grid placed over each photomicrograph. Using a random number generator, five sections from each grid were selected for counting, giving a total of 25 fields per rat. Capillary density was expressed as the number per mm². Only sections oriented perpendicular to the vessels were counted. Arteriolar density was counted in a similar manner using a magnification of 200× because there are far fewer arterioles than capillaries. The investigator reading the capillary and arteriolar density was blinded to treatment group and time of sacrifice.

Echocardiography. Echocardiographic measurements of LV mass and fractional area shortening were made from digital images acquired with a 12 MHz broadband transducer (S12 probe, Philips Ultrasound, Bothell, Wash.). LV mass was calculated by area-length method as follows:

LV mass=1.05{[5/6A₁(L+t)]−[5/6A₂(L)]},

where A₁=epicardial area and A₂=endocardial area obtained from short-axis views at end-diastole; L=left ventricle (LV) length from the LV apex to the middle of the mitral annulus from long-axis views at end-diastole; t=myocardial thickness back calculated from the short-axis cavity area.
Fractional area shortening was evaluated from the following formula:

FS=(LVEDA−LVESA)/LVEDA,

where LVEDA=left ventricle end-diastolic area (cm²) and LVESA=left ventricle end-systolic area (cm²).

Data analysis. Data was analyzed with Statview software (SAS, Cary, N.C.). The results are expressed as mean±one standard deviation. Differences were analyzed by ANOVA with Fisher's post-hoc test and considered significant at p<0.05.

Manufacture of plasmid-containing lipid-stabilized microbubbles. Lipid-stabilized microbubbles were prepared as previously described by the present inventors.^{54,55} Briefly, a solution of DPPC (1,2-dipalmitoyl-sn-glycero-3-phosphatidylcholine, Sigma, St. Louis, Mo.) 2.5 mg/ml; DPPE (1,2-dipalmitoyl-sn-glycero-3-phosphatidylethanolamine, Sigma, St. Louis, Mo.) 0.5 mg/ml; and 10% glycerol was placed in 1.5 ml clear vials; the remaining headspace was filled with the perfluoropropane gas (Air Products, Inc, Allentown, Pa.). Each vial was incubated at room temperature for 30 min and then mechanically shaken for 20 seconds by a dental amalgamator (Vialmix™, Bristol-Myers Squibb Medical Imaging, N. Billerica, Mass.). The lipid-stabilized microbubbles appear as a milky white suspension floating on the top of a layer of liquid. The liquid subnatant was discarded and the mean diameter and concentration of the microbubbles in the upper layer were measured by a particle counter (Beckman Coulter Multisizer III). Cationic liposomes containing plasmid DNA were made with 50 μl of cationic liposome solution (lipofectamine 2000, Invitrogen) mixed with 2 mg of plasmid DNA and incubated for 15 minutes at room temperature. This forms nanosphere-sized cationic liposome complexes encapsulating the plasmid DNA.⁷⁹ Microbubbles with the cationic liposome-plasmid complexes were made as above by adding 50 μl of liposomes to 250 μl of the phospholipid-coated microbubbles and shaking in the amalgamator for 20 seconds at room temperature with perfluoropropane gas filling the head space of the vial.

Plasmid constructs and DNA preparation. Plasmids expressing the hVEGF₁₆₅ gene under the enhanced CMV promoter with an intron were made as follows: total mRNA was extracted from a healthy volunteer blood with a QIAamp Blood kit (Qiagen Inc, Valencia, Calif.) according to the manufacturer's instructions. And then mRNA was reversed into cDNA with a SuperScript first-strand synthesis system for RT-PCR kit (Invitrogen). A full length cDNA of the hVEGF₁₆₅ cDNA was PCR amplified by using the following PCR primers that contain a restriction site at the 5′ ends (the restriction sites are underlined):

primer 1 (XhoI)
(SEQ ID NO.: 14)
5'-TTCCTCGAGAATGAACTTTCTGCTGCTGTCTTG-3';
primer 2 (Smal)
(SEQ ID NO.: 15)
5'-AAACCCGGGTCACCGCCTCGGCTTGTCA-3'.

The product was confirmed by sequencing. The DNA was digested with XhoI and SmaI and then ligated into the corresponding sites of pCI-neo (Promega). Cloning, isolation and purification of this plasmid were performed by standard procedures,⁸⁰ and once again sequenced to confirm that no artifactual mutations were present.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

In the claims, all transitional phrases such as “comprising,” “including,” “carrying,” “having,” “containing,” “involving,” and the like are to be understood to be open-ended, i.e., to mean including but not limited to. Only the transitional phrases “consisting of” and “consisting essentially of,” respectively, shall be closed or semi-closed transitional phrases.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES CITED

1. King, H., Aubert, R. E. & Herman, W. H. Global burden of diabetes, 1995-2025: prevalence, numerical estimates, and projections. Diabetes Care 21, 1414-31 (1998).
2. Yamaoka, T. Gene therapy for diabetes mellitus. Curr Mol Med 1, 325-37 (2001).
3. Becker, T., Beltran del Rio, H., Noel, R. J., Johnson, J. H., & Newgard, C. B. Overexpression of Hexokinase I in Isolated Islets of Langerhans via Recombinant Adenovirus: Enhancement of Glucose Metabolism and Insulin Secretion at Basal but not Stimulatory Glucose Levels. J. Biol. Chem. 269, 21234-21238 (1994).
4. Flotte T, Agarwal A, Wang J, Song S, Fenjves E S, Inverardi L, Chesnut K, Afione S, Loiler S, Wasserfall C, Kapturczak M, Ellis T, Nick H, Atkinson M: Efficient ex vivo transduction of pancreatic islet cells with recombinant adenoassociated virus vectors. Diabetes 50:515-520, 2001
5. Isner, J. M. Myocardial gene therapy. Nature 415, 234-239 (2002).
6. Shohet, R. V. et al. Echocardiographic destruction of albumin microbubbles directs gene delivery to the myocardium. Circulation 101, 2554-2556 (2000).
7. Chen, S. Y., Shohet, R. V., Bekeredjian, R., Frenkel, P. A. & Grayburn, P. A. Optimization of ultrasound parameters for cardiac gene delivery of adenoviral and plasmid deoxyribonucleic acid by ultrasound-targeted microbubble destruction. J. Am. Coll. Cardiol. 42, 301-8 (2003).
8. Bekeredjian, R., Chen, S-Y., Frenkel, P., Grayburn, P. A. & Shohet, R. V. Ultrasound targeted microbubble destruction can repeatedly direct highly specific plasmid expression to the heart. Circulation 108, 1022-26 (2003).
9. Korpanty G. et al. Targeting of VEGF-mediated angiogenesis to rat myocardium using ultrasonic destruction of microbubbles. Gene Ther. In press (2005).
10. Bekeredjian R., Grayburn P. A., & Shohet R. V. Use of ultrasound contrast agents for gene or drug delivery in cardiovascular medicine. J. Am. Coll. Cardiol. 45, 329-35 (2005).
11. Frenkel, P. A., Chen, S-Y., Thai, T., Shohet, R. V. & Grayburn, P. A. DNA-loaded albumin microbubbles enhance ultrasound-mediated transfection in vitro. Ultrasound Med. Biol. 28, 817-22 (2002).
12. Wickham, T. J. Targeting adenovirus. Gene Ther. 2000 7, 110-114 (2000).
13. Reynolds, P. N. et al. Combined transductional and transcriptional targeting improves the specificity of transgene expression in vivo. Nat. Biotechnol. 19, 838-842 (2001).
14. Boast, K. et al. Characterization of physiologically regulated vectors for the treatment of ischemic disease. Hum. Gene Ther. 10, 2197-2208 (1999).
15. Shah, A. S. et al. Intracoronary adenovirus-mediated delivery and overexpression of the beta(2)-adrenergic receptor in the heart: prospects for molecular ventricular assistance. Circulation 101, 408-414 (2000).
16. Logeart, D. et al. How to optimize in vivo gene transfer to cardiac myocytes: mechanical or pharmacological procedures? Hum. Gene Ther. 12, 1601-1610 (2001).
17. French, B. A. et al. Direct in vivo gene transfer into porcine myocardium using replication-deficient adenoviral vectors. Circulation 90, 2414-2424 (1994).
18. Tio, R. A. et al. Intramyocardial gene therapy with naked DNA encoding vascular endothelial growth factor improves collateral flow to ischemic myocardium. Hum. Gene Ther. 10, 2953-2960 (1999).
19. Vincent, K. A. et al. Angiogenesis is induced in a rabbit model of hindlimb ischemia by naked DNA encoding an HIF-1alpha/VP16 hybrid transcription factor. Circulation 102, 2255-2261 (2000).
20. Epstein P. N., Boschero A. C., Atwater I., Cai X., & Overbeek P. A. Expression of yeast hexokinase in pancreatic beta cells of transgenic mice reduces blood glucose, enhances insulin secretion, and decreases diabetes. Proc. Natl. Acad. Sci. U.S.A. 89, 12038-12042 (1992).
21. Yamamoto, K. et al. Overexpression of PACAP in transgenic mouse pancreatic beta-cells enhances insulin secretion and ameliorates streptozotocin-induced diabetes. Diabetes 52, 1155-62 (2003).
22. Hara, M. et al. Transgenic mice with green fluorescent protein-labeled pancreatic beta-cells. Am. J. Physiol. Endocrinol. Metab. 284, E177-83 (2003).
23. Marron, M. P., Graser, R. T., Chapman, H. D. & Serreze, D. V. Functional evidence for the mediation of diabetogenic T cell responses by HLA-A2.1 MHC class I molecules through transgenic expression in NOD mice. Proc. Natl. Acad. Sci. U.S.A. 99, 13753-8 (2002).
24. Cebrian, A. et al. Overexpression of parathyroid hormone-related protein inhibits pancreatic beta-cell death in vivo and in vitro. Diabetes 51, 3003-13 (2002).
25. Hussain, M. A., Miller, C. P, & Habener, J. F. Brn-4 transcription factor expression targeted to the early developing mouse pancreas induces ectopic glucagon gene expression in insulin-producing beta cells. J. Biol. Chem. 2002 277, 16028-32 (2002).
26. Tuttle, R. L et al. Regulation of pancreatic beta-cell growth and survival by the serine/threonine protein kinase Akt1/PKBalpha. Nat. Med. 7, 1133-7 (2001).
27. Thorens, B., Guillam, M. T., Beermann, F., Burcelin, R. & Jaquet, M. Transgenic reexpression of GLUT1 or GLUT2 in pancreatic beta cells rescues GLUT2-null mice from early death and restores normal glucose-stimulated insulin secretion. J. Biol. Chem. 275, 23751-8 (2000).
28. Stellrecht, C. M., DeMayo, F. J., Finegold, M. J., Tsai, M. J. Tissue-specific and developmental regulation of the rat insulin II gene enhancer, RIPE3, in transgenic mice. J. Biol.
Chem. 272, 3567-72 (1997).
29. Pawelczyk, T., Podgorska, M. & Sakowicz. M. The effect of insulin on expression level of nucleoside transporters in diabetic rats. Mol. Pharmacol. 63, 81-8 (2003).
30. Zhang, S. L. et al. Insulin inhibits dexamethasone effect on angiotensinogen gene expression and induction of hypertrophy in rat kidney proximal tubular cells in high glucose. Endocrinology 143, 4627-35 (2002).
31. Porte, D. Jr. & Kahn, S. E. Beta-cell dysfunction and failure in type 2 diabetes: potential mechanisms. Diabetes 50 (Suppl 1), S160-3 (2001).
32. Shimabukuro, M. et al. Lipoapoptosis in beta-cells of obese prediabetic fa/fa rats. J. Biol. Chem. 273, 32487-90 (1998).
33. Unger, R. H. & Zhou, Y. T. Lipotoxicity of beta-cells in obesity and in other causes of fatty acid spillover. Diabetes 50 (Suppl), S118-21 (2001).
34. Maedler, K. et al. Glucose-induced beta cell production of IL-1beta contributes to glucotoxicity in human pancreatic islets. J. Clin. Invest. 110, 851-60 (2002).
35. Fajans, S. S., Bell, G. I. & Polonsky, K. S. Molecular mechanisms and clinical pathophysiology of maturity-onset diabetes of the young. N. Engl. J. Med. 345, 971-80 (2001).
36. Zeng, Y., Yi, R. & Cullen, B. R. MicroRNAs and small interfering RNAs can inhibit mRNA expression by similar mechanisms. Proc. Natl. Acad. Sci. U.S.A. 100, 9779-84 (2003).
37. Bain, J., Schisler, J. C., Takeuchi, K., Newgard, C. B., & Becker, T. C. An adenovirus vector for efficient RNAi-mediated suppression of target genes in insulinoma cells and pancreatic islets of Langherhans. Diabetes 53, 2190-2194 (2004).
38. Hammond H K, McKirnan M D. Angiogenic gene therapy for heart disease: a review of animal studies and clinical trials. Cardiovasc Res 2001; 49:561-567.
39. Simons M, et al. Clinical trials in coronary angiogenesis: issues, problems, consensus: An expert panel summary. Circulation 2000; 102: E73-E86.
40. Pecher P, Schumacher B A. Angiogenesis in ischemic human myocardium: clinical results after 3 years. Ann Thorac Surg 2000; 69: 1414-1419.
41. Simons M, Ware J A. Therapeutic angiogenesis in cardiovascular disease. Nat Rev Drug Discov 2003; 2: 863-871.
42. Rosengart T K, et al. Six-month assessment of a Phase I trial of angiogenic gene therapy for the treatment of coronary artery disease using direct intramyocardial administration of an adenovirus vector expressing the VEGF121 cDNA. Ann Surgery 1999; 230: 466-472.
43. Udelson J E, et al. Therapeutic angiogenesis with recombinant fibroblast growth factor-2 improves stress and rest myocardial perfusion abnormalities in patients with severe symptomatic chronic coronary artery disease. Circulation 2000; 102: 1605-1610.
44. Henry T D, et al. The VIVA trial: Vascular endothelial growth factor in Ischemia for Vascular Angiogenesis. Circulation 2003; 107: 1359-1365.
45. Simons M, et al. Pharmacological treatment of coronary artery disease with recombinant fibroblast growth factor-2: double-blind, randomized, controlled clinical trial. Circulation 2002; 105: 788-793.
46. Laham R J, et al. Local perivascular delivery of basic fibroblast growth factor in patients undergoing coronary bypass surgery: results of a phase I randomized, double-blind, placebo-controlled trial. Circulation 1999; 100: 1865-1871.
47. Ruel M, et al. Long-term effects of surgical angiogenic therapy with fibroblast growth factor 2 protein. J Thorac Cardiovasc Surg 2002; 124: 28-34.
48. Lederman R J, et al. Therapeutic angiogenesis with recombinant fibroblast growth factor-2 for intermittent claudication (the TRAFFIC study): a randomised trial. Lancet 2002; 359: 2053-2058.
49. Grines C L, et al. Angiogenic gene therapy (AGENT) trial in patients with stable angina pectoris. Circulation 2002; 105: 1291-1297.
50. Losordo D W, et al. Phase 1/2 placebo-controlled, double-blind, dose-escalating trial of myocardial vascular endothelial growth factor 2 gene transfer by catheter delivery in patients with chronic myocardial ischemia. Circulation 2002; 105: 2012-2018.
51. Hedman M, et al. Safety and feasibility of catheter-based local intracoronary vascular endothelial growth factor gene transfer in the prevention of postangioplasty and in-stent restenosis and in the treatment of chronic myocardial ischemia: phase II results of the Kuopio Angiogenesis Trial (KAT). Circulation 2003; 107: 2677-2683.
52. Shohet R V, et al. Echocardiographic destruction of albumin microbubbles directs gene delivery to the myocardium. Circulation 2000; 101: 2554-2556.
53. Chen S Y, et al. Optimization of ultrasound parameters for cardiac gene delivery of adenoviral or plasmid deoxyribonucleic acid by ultrasound-targeted microbubble destruction. J Am Coll Cardiol 2003; 42: 301-308.
54. Bekeredjian R, et al. Ultrasound targeted microbubble destruction can repeatedly direct highly specific plasmid expression to the heart. Circulation 2003; 108: 1022-1026.
55. Dor Y, et al. Conditional switching of VEGF provides new insights into adult neovascularization and pro-angiogenic therapy. EMBO J 2002; 21: 1939-1947.
56. D'Amore P A. Capillary growth: a two-cell system. Semin Cancer Biol 1992; 3: 49-56.
57. Carmeliet P, Collen D. Genetic analysis of blood vessel formation. Role of endothelial versus smooth muscle cells. Trends Cardiovasc Med 1997; 7: 271-281.
58. Grosskreutz C L, et al. Vascular endothelial growth factor-induced migration of vascular smooth muscle cells in vitro. Microvasc Res 1999; 58: 128-136.
59. Carmeliet P. Mechanisms of angiogenesis and arteriogenesis. Nat Med 2000; 6: 389-395.
60. Song J, Ming Q I, Kaul S, Price R J. Stimulation of arteriogenesis in skeletal muscle by Microbubbles destruction with ultrasound. Circulation 2002; 106: 1550-1555.
61. Greenleaf W J, et al. Artificial cavitation nuclei significantly enhance acoustically induced cell transfection. Ultrasound Med Biol 1998; 24: 587-595.
62. Miller D L, Gies R A. The interaction of ultrasonic heating and cavitation in vascular bioeffects on mouse intestine. Ultrasound Med Biol 1998; 24: 123-128.
63. Wu J, Ross J P, Chiu J F. Reparable sonoporation generated by microstreaming. J Acoust Soc Am 2002; 111: 1460-1464.
64. Kondo T, Misik V, Riesz P. Effect of gas-containing microspheres and echo contrast agents on free radical formation by ultrasound. Free Radic Biol Med 1998; 25: 605-612.
65. Skyba D M, et al. Direct in vivo visualization of intravascular destruction of microbubbles by ultrasound and its local effects on tissue. Circulation 1998; 98: 290-293.
66. Price R J, Skyba D M, Kaul S, Skalak T C. Delivery of colloidal particles and red blood cells to tissue through microvessel ruptures created by targeted microbubble destruction with ultrasound. Circulation 1998; 98: 1264-1267.
67. Croll S D., et al. VEGF-mediated inflammation precedes angiogenesis in adult brain. Exp Neurol 2004; 187: 388-402.
68. Mor F, Quintana F J, Cohen I R. Angiogenesis-inflammation cross-talk: vascular endothelial growth factor is secreted by activated T cells and induces Th1 polarization. J Immunol 2004; 172: 4618-23.
69. Chavakis T, et al. Regulation of neovascularization by human neutrophil peptides (alpha-defensins): a link between inflammation and angiogenesis. FASEB J 2004; epub ahead of print.
70. Chen S Y, et al. Bioeffects of myocardial contrast microbubble destruction by echocardiography. Echocardiography 2002; 19: 495-500.
71. Bekeredjian R, et al. Effects of ultrasound targeted microbubble destruction on cardiac gene expression. J Ultrasound Med Biol 2004; 30: 539-43.
72. Hershey J C, et al. Revascularization in the rabbit hindlimb: dissociation between capillary sprouting and arteriogenesis. Cardiovasc Res 2001; 49: 618-625.
73. Helisch A, Schaper W. Arteriogenesis: the development and growth of collateral arteries. Microcirculation 2003; 10: 83-97.
74. Ware J A, Simons M. Angiogenesis in ischemic heart disease. Nature Med 1997; 3: 158-164.
75. Losordo D W, Dimmeler S. Therapeutic angiogenesis and vasculogenesis for ischemic disease. Part I. Angiogenic cytokines. Circulation 2004; 109: 2487-2491.
76. Moulton K S, et al. Inhibition of plaque neovascularization reduces macrophage accumulation and progression of advanced atherosclerosis. Proc Natl Acad Sci USA 2003; 100: 4736-4741.
77. Belgore F, et al. Localisation of members of the vascular endothelial growth factor (VEGF) family and their receptors in human atherosclerotic arteries. J Clin Pathol 2004; 57: 266-272.
78. Khurana R, et al. Angiogenesis-dependent and independent phases of intimal hyperplasia. Circulation 200; 110:2436-2443.
79. Templeton N S, et al. Improved DNA: liposome complexes for increased systemic delivery and gene expression. Nat Biotechnol 1997; 15: 647-652.
80. Maniatis T, Fritsch E F, Sambrook J. Extraction and Purification of plasmid DNA. In: Sambrook J, (Ed) Molecular Cloning Manual. Cold Spring Harbor Press, Cold Spring Harbor, N. Y., 1989, pp. 1.38-1.39.

1.-34. (canceled)

35. A method of preparing one or more bioactive agents, comprising the steps of:
providing the one or more bioactive agents encapsulated within or attached to a liposome to produce pre-loaded liposomes; and
loading the pre-loaded liposomes onto neutrally charged microbubbles to produce microbubble-encapsulated bioactive agents.

36. The method of claim 35, further comprising the steps:
contacting a target organ or tissue of an individual with the microbubble-encapsulated bioactive agents; and
selectively releasing the bioactive agents at the target organ or tissue by exposing the microbubble at the target with ultrasound.

37. The method of claim 35, wherein the bioactive agent comprises a nucleic acid segment under the control of a tissue-specific promoter.

38. The method of claim 35, wherein the bioactive agent comprises a nucleic acid segment under the control of an activatable promoter.

39. The method of claim 35, wherein the bioactive agent comprises a nucleic acid segment under the control of an activatable promoter that drives expression of a gene that causes apoptosis.

40. The method of claim 35, wherein the bioactive agent comprises a nucleic acid segment that encodes a gene selected from the group consisting of hormone, growth factor, enzyme, apolipoprotein clotting factor,—tumor suppressor, tumor antigen, viral protein, bacterial surface protein, and parasitic cell surface protein.

41. The method of claim 35, wherein the microbubbles are disposed in a pharmaceutically acceptable vehicle.

42. The method of claim 35, wherein the bioactive agent comprises an expressible gene selected from the group consisting of p53, p16, p21, MMAC1, p′73, zac1, C-CAM, BRCAI, Rb, Harakiri, Ad E1 B, ICE-CED3 protease, IL-2, IL-3, IL-4, IL-5, IL-6, IL-7, IL-8, IL-9, IL-10, IL-11, IL-12, IL-13, IL-14, IL-15, TNF, GMCSF, β-interferon, γ-interferon, VEGF, EGF, PDGF, CFTR, EGFR, VEGFR, IL-2 receptor, estrogen receptor, Bcl-2 or Bcl-xL, ras, myc, neu, raf, erb, src, fins, jun, trk, ret, gsp, hst, abl, p53, p16, p21, MMAC1, p73, zacl, BRCAI, BRCAII, Rb, growth hormone, nerve growth factor, insulin, adrenocorticotropic hormone, parathormone, follicle-stimulating hormone, luteinizing hormone and thyroid stimulating hormone.

43. The method of claim 35, wherein the bioactive agent comprises a nucleic acid segment under the control of a promoter selected from the group consisting of CMV IE, LTR, SV 40 IE, HSV tk, β-actin, insulin, human globin α, human globin β and human globin γ promoter.

44. The method of claim 36, wherein the ultrasound is applied in a pulsed and focused mode.

45. The method of claim 36, wherein the ultrasound is applied in ultraharmonic mode.

46. The method of claim 35, wherein the microbubbles comprise a biodegradable polymer.

47. The method of claim 35, wherein the microbubbles comprise a biocompatible amphiphilic material.

48. The method of claim 35, wherein the microbubbles comprise an outer shell comprising an outer layer of biologically compatible amphiphilic material and an inner layer of a biodegradable polymer.

49. The method of claim 47, wherein the amphiphilic material is selected from the group consisting of collagen, gelatin, albumin, and globulin.

50. The method of claim 35, wherein the liposomes comprise 1,2-dipalmitoyl-sn-glycero-3-phosphatidylcholine and 1,2-dipalmitoyl-sn-glycero-3-phosphatidylethanolamine glycerol mixed with a plasmid.


SEQUENCE LISTING (15 sequences)

SEQ ID NO: 1 - DNA, 27 bp, artificial sequence
  primer
caactcgaggctgagctaagaatccag

SEQ ID NO: 2 - DNA, 27 bp, artificial sequence
  primer
gcagaattcctgcttgctgatggtcta

SEQ ID NO: 3 - DNA, 36 bp, artificial sequence
  primer
aaagaattcatgatcgccgcgcaactactggcctat

SEQ ID NO: 4 - DNA, 35 bp, artificial sequence
  primer
aaagcggccgcttaggcgatcgaagggtctcctct

SEQ ID NO: 5 - DNA, 21 bp, Artificial sequence
  primer
gagttcatgcgcttcaaggtg

SEQ ID NO: 6 - DNA, 21 bp, Artificial sequence
  primer
ttggagtccacgtagtagtag

SEQ ID NO: 7 - DNA, 21 bp, Artificial sequence
  primer
gagttcatgcgcttcaaggtg

SEQ ID NO: 8 - DNA, 21 bp, Artificial sequence
  primer
ttggagtccacgtagtagtag

SEQ ID NO: 9 - DNA, 21 bp, Artificial sequence
  primer
gatggtgatgtcctcgttgtg

SEQ ID NO: 10 - DNA, 21 bp, Artificial sequence
  primer
ggaggagggcagaatcatcac

SEQ ID NO: 11 - DNA, 22 bp, Artificial sequence
  primer
cgctctgagcaaggcccacagg

SEQ ID NO: 12 - DNA, 24 bp, Artificial sequence
  primer
acagaaggggagcagaaagcccat

SEQ ID NO: 13 - DNA, 22 bp, Artificial sequence
  primer
cgctctgaccaaggctcacagt

SEQ ID NO: 14 - DNA, 33 bp, Artificial sequence
  primer
ttcctcgagaatgaactttctgctgctgtcttg

SEQ ID NO: 15 - DNA, 28 bp, Artificial sequence
  primer
aaacccgggtcaccgcctcggcttgtca